(19) United States
(12) Patent Application Publication
(10) Pub. No.: US 2023/0338428 A1
(43) Pub. Date: Oct. 26, 2023

(54) ALLOGENIC UMBILICAL CORD STEM CELLS FOR TREATING SEVERE RESPIRATORY CONDITIONS

(71) Applicant: Amit Patel, Shalimar, FL (US)

(72) Inventor: Amit Patel, Shalimar, FL (US)

(21) Appl. No.: 18/025,388

(22) PCT Filed: Sep. 8, 2021
(86) PCT No.: PCT/US2021/049535

§ 371 Date: Mar. 8, 2023

Related U.S. Application Data
(60) Provisional application No. 63/075,780, filed on Sep. 8, 2020.

(51) Int. Cl. A61K 35/28; A61K 35/32; A61K 35/34; A61K 9/00; A61P 11/00

(52) U.S. Cl. CPC A61K 35/28; A61K 35/32; A61K 35/34; A61K 9/0019; A61P 11/00

(57) ABSTRACT

Method of treating a respiratory condition in a subject by infusing a composition comprising stem or progenitor cells to a subject having a respiratory condition, wherein the stem or progenitor cells express at least three cell markers selected from CD29, CD73, CD90, CD166, SSEA4, CD9, CD44, CD146, or CD105 and wherein the stem or progenitor cells do not express at least five cell markers selected from the group consisting of NANOG, CD45, CD34, CD14, CD79, CD106, CD86, CD80, CD19, CD117, Stro-1, or HLA-DR.

### BACKGROUND

Certain pathogens, such as bacteria, viruses, fungi, or parasites, to name a few, are capable of infecting a human and causing an infectious condition. Some pathogens live in and on the human body, becoming infectious at times when the human's immune system is compromised or otherwise weakened. Other pathogens, however, encounter a subject by chance and infect through direct infiltration through the eyes, mouth, nose, etc. In some cases, chance encounters can be opportunities whereby the pathogen passed from one subject to another. In other cases, chance encounters may be animal or insect transmission, consumption of contaminated food or water that has been exposed to pathogens.

Pathogens can infect various tissues/organs of the human body. The symptoms that a human subject experiences when infected can vary depending on the tissue or organ type that has been infected.

### BRIEF DESCRIPTION OF THE DRAWINGS

FIG. 1 shows an image of a histological section of umbilical cord identifying the subepithelial layer in accordance with one aspect of the present disclosure.

FIG. 2A shows explant of cells migrating from the subepithelial layer and karyotyping of cells in accordance with another aspect of the present disclosure.

FIG. 2B shows explant of cells migrating from the subepithelial layer and karyotyping of cells in accordance with another aspect of the present disclosure.

FIG. 2C shows karyotyping of cells in accordance with another aspect of the present disclosure

FIG. 3A shows FACS analysis of cell determinant markers expressed by cells or stem cells derived from umbilical cord in accordance with another aspect of the present disclosure.

FIG. 3B shows FACS analysis of cell determinant markers expressed by cells or stem cells derived from umbilical cord in accordance with another aspect of the present disclosure.

FIG. 3C shows FACS analysis of cell determinant markers expressed by cells or stem cells derived from umbilical cord in accordance with another aspect of the present disclosure.

FIG. 3D shows FACS analysis of cell determinant markers expressed by cells or stem cells derived from umbilical cord in accordance with another aspect of the present disclosure.

FIG. 3E shows FACS analysis of cell determinant markers expressed by cells or stem cells derived from umbilical cord in accordance with another aspect of the present disclosure.

FIG. 3F shows FACS analysis of cell determinant markers expressed by cells or stem cells derived from umbilical cord in accordance with another aspect of the present disclosure.

FIG. 3G shows FACS analysis of cell determinant markers expressed by cells or stem cells derived from umbilical cord in accordance with another aspect of the present disclosure.

FIG. 3H shows FACS analysis of cell determinant markers expressed by cells or stem cells derived from umbilical cord in accordance with another aspect of the present disclosure.

FIG. 3I shows FACS analysis of cell determinant markers expressed by cells or stem cells derived from umbilical cord in accordance with another aspect of the present disclosure.

FIG. 3J shows FACS analysis of cell determinant markers expressed by cells or stem cells derived from umbilical cord in accordance with another aspect of the present disclosure.

FIG. 3K shows FACS analysis of cell determinant markers expressed by cells or stem cells derived from umbilical cord in accordance with another aspect of the present disclosure.

FIG. 3L shows FACS analysis of cell determinant markers expressed by cells or stem cells derived from umbilical cord in accordance with another aspect of the present disclosure.

FIG. 3M shows FACS analysis of cell determinant markers expressed by cells or stem cells derived from umbilical cord in accordance with another aspect of the present disclosure.

FIG. 3N shows FACS analysis of cell determinant markers expressed by cells or stem cells derived from umbilical cord in accordance with another aspect of the present disclosure.

FIG. 3O shows FACS analysis of cell determinant markers expressed by cells or stem cells derived from umbilical cord in accordance with another aspect of the present disclosure.

FIG. 4A shows images of RT-PCR analysis of RNA extracted from cells or stem cells derived from umbilical cord in accordance with another aspect of the present disclosure.

FIG. 4B shows images of immunocytochemical staining of cells in accordance with another aspect of the present disclosure.

FIG. 4C shows images of immunocytochemical staining of cells in accordance with another aspect of the present disclosure.

FIG. 4D shows images of immunocytochemical staining of cells in accordance with another aspect of the present disclosure.

FIG. 5A shows images of culture of cells or stem cells derived from umbilical cord tissue in semi-solid PRP matrix or PL Lysate in accordance with another aspect of the present disclosure.

FIG. 5B shows images of culture of cells or stem cells derived from umbilical cord tissue in semi-solid PRP matrix or PL Lysate in accordance with another aspect of the present disclosure.

FIG. 6A shows extracellular exosome size analysis in accordance with another aspect of the present disclosure.

FIG. 6B shows an SEM of exosomes in accordance with another aspect of the present disclosure.

FIG. 6C shows CD63 expression of exosomes produced from cells or stem cells derived from umbilical cord in accordance with another aspect of the present disclosure.

FIG. 6D shows CD63 expression of exosomes produced from cells or stem cells derived from umbilical cord in accordance with another aspect of the present disclosure.

FIG. 7A shows images demonstrating differentiation of umbilical cord tissue into adipogeneic lineages in accordance with another aspect of the present disclosure.

FIG. 7B shows images demonstrating differentiation of umbilical cord tissue into adipogeneic lineages in accordance with another aspect of the present disclosure.

FIG. 7C shows images demonstrating differentiation of umbilical cord tissue into adipogeneic lineages in accordance with another aspect of the present disclosure.

FIG. 7D shows images demonstrating differentiation of umbilical cord tissue into adipogeneic lineages in accordance with another aspect of the present disclosure.

FIG. 8A shows images demonstrating differentiation of umbilical cord tissue into osteogenic lineages in accordance with another aspect of the present disclosure.

FIG. 8B shows images demonstrating differentiation of umbilical cord tissue into osteogenic lineages in accordance with another aspect of the present disclosure.

FIG. 8C shows images demonstrating differentiation of umbilical cord tissue into osteogenic lineages in accordance with another aspect of the present disclosure.

FIG. 8D shows images demonstrating differentiation of umbilical cord tissue into osteogenic lineages in accordance with another aspect of the present disclosure.

FIG. 9A shows an image demonstrating differentiation of umbilical cord tissue into Chondrogenic lineages in accordance with another aspect of the present disclosure.

FIG. 9B shows an image demonstrating differentiation of umbilical cord tissue into Chondrogenic lineages in accordance with another aspect of the present disclosure.

FIG. 10A shows an image demonstrating differentiation of umbilical cord tissue into cardiogenic lineages in accordance with another aspect of the present disclosure.

FIG. 10B shows an image demonstrating differentiation of umbilical cord tissue into cardiogenic lineages in accordance with another aspect of the present disclosure.

FIG. 10C shows an image demonstrating differentiation of umbilical cord tissue into cardiogenic lineages in accordance with another aspect of the present disclosure.

FIG. 10D shows an image demonstrating differentiation of umbilical cord tissue into cardiogenic lineages in accordance with another aspect of the present disclosure.

FIG. 11 shows the details of the UC-MSC potency assay in accordance with another aspect of the present disclosure.

FIG. 12A shows SAE-free survival was significantly improved in the UC-MSC treatment group compared to the Control group in accordance with another aspect of the present disclosure.

FIG. 12B shows SAE-free survival was significantly improved in the UC-MSC treatment group compared to the Control group in accordance with another aspect of the present disclosure.

FIG. 12C shows time to recovery was significantly improved in the UC-MSC treatment group compared to the Control group in accordance with another aspect of the present disclosure.

FIG. 13 shows a calibration curve (Four Parameter Logistic Curve) based on recombinant sTNFR2 in accordance with one aspect of the present disclosure.

FIG. 14 shows a calibration curve (Four Parameter Logistic Curve) based on recombinant sTNFR2 in accordance with one aspect of the present disclosure.

FIG. 15 provides a table that shows that batches of UC-MSC, when properly cryopreserved, maintain potency and stability in accordance with another aspect of the present disclosure.

FIG. 16 provides a table that shows that batches of UC-MSC, when properly cryopreserved, maintain potency and stability in accordance with another aspect of the present disclosure.

FIG. 17 provides a table that shows that batches of UC-MSC, when properly cryopreserved, maintain potency and stability in accordance with another aspect of the present disclosure.

FIG. 18 shows that sTNFR2 was increased in patients of the UC-MSC treatment group compared to patients in the control group at day 6 in accordance with another aspect of the present disclosure.

### DESCRIPTION OF EMBODIMENTS

Although the following detailed description contains many specifics for the purpose of illustration, a person of ordinary skill in the art will appreciate that many variations and alterations to the following details can be made and are considered included herein. Accordingly, the following embodiments are set forth without any loss of generality to, and without imposing limitations upon, any claims set forth. It is also to be understood that the terminology used herein is for describing particular embodiments only, and is not intended to be limiting. Unless defined otherwise, all technical and scientific terms used herein have the same meaning as commonly understood by one of ordinary skill in the art to which this disclosure belongs. Also, the same reference numerals in appearing in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of layouts, distances, network examples, etc., to provide a thorough understanding of various embodiments. One skilled in the relevant art will recognize, however, that such detailed embodiments do not limit the overall concepts articulated herein, but are merely representative thereof. One skilled in the relevant art will also recognize that the technology can be practiced without one or more of the specific details, or with other methods, components, layouts, etc. In other instances, well-known structures, materials, or operations may not be shown or described in detail to avoid obscuring aspects of the disclosure.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term in this written description, like “comprising” or “including,” it is understood that direct support should be afforded also to “consisting essentially of” language as well as “consisting of” language as if stated explicitly and vice versa.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a given term, metric, value, range endpoint, or the like. The degree of flexibility for a particular variable can be readily determined by one skilled in the art. However, unless otherwise expressed, the term “about” generally provides flexibility of less than 1%, and in some cases less than 0.01%. It is to be understood that, even when the term “about” is used in the present specification in connection with a specific numerical value, support for the exact numerical value recited apart from the “about” terminology is also provided.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of phrases including “an example” or “an embodiment” in various places throughout this specification are not necessarily all referring to the same example or embodiment.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” and the like refer to a property of a device, component, or activity that is measurably different from other devices, components, or activities in a surrounding or adjacent area, in a single device or in multiple comparable devices, in a group or class, in multiple groups or classes, or as compared to the known state of the art.

A “stem cell” refers to an undifferentiated cell capable of self-renewal, or in other words, the ability to generate at least one identical copy of the original cell, differentiation at the single cell level into one or more specialized cell types, and culture expansion. More specialized types of stem cells are subclassified according to their developmental potential as totipotent, pluripotent, multipotent, and oligo/unipotent.

A “progenitor cell” is a specialized stem cell capable of self-renewal, and differentiation into more mature cells, but is committed to a developmental lineage (e.g., hematopoietic progenitors are committed to the blood lineage; myeloid progenitors are committed to the myeloid lineage; lymphoid progenitors are committed to the lymphoid lineage).

A “stem cell” refers to an undifferentiated cell capable of self-renewal, or in other words, the ability to generate at least one identical copy of the original cell, differentiation at the single cell level into one or more specialized cell types, and culture expansion. More specialized types of stem cells are subclassified according to their developmental potential as totipotent, pluripotent, multipotent, and oligo/unipotent.

A “progenitor cell” is a specialized stem cell capable of self-renewal, and differentiation into more mature cells, but is committed to a developmental lineage (e.g., hematopoietic progenitors are committed to the blood lineage; myeloid progenitors are committed to the myeloid lineage; lymphoid progenitors are committed to the lymphoid lineage).

“Autologous,” as used herein, refers to cells from the same subject.

The term “allogeneic” refers to cells of the same species that are genetically different in one or more genetic loci. As one example, allogenic cells include cells that transplanted from one animal to another non-identical animal of the same species.

“Administering” a composition may be accomplished by oral administration, injection, infusion, parenteral, intravenous, mucosal, sublingual, intramuscular, intradermal, intranasal, intraperitoneal, intraarterial, subcutaneous absorption or by any method in combination with other known techniques. In one example, the composition can be administered systemically. In another example, the composition can be administered by infusion or direct injection. In yet another example, the composition can be administered intravenously, intraarterially, or intraperitoneally.

The term “subject” as used herein includes, but is not limited to, humans and non-human vertebrates such as wild, domestic, and farm animals. In some embodiments, the term refers to non-human animals, such as dogs, cats, birds, mice, rats, rabbits, guinea pigs, hamsters, gerbils, goats, sheep, bovines, horses, camels, non-human primates, etc. In some embodiments, the term refers to humans.

An initial overview of embodiments is provided below, and specific embodiments are then described in further detail. This initial summary is intended to aid readers in understanding the disclosure more quickly and is not intended to identify key or essential technological features, nor is it intended to limit the scope of the claimed subject matter.

Various respiratory conditions in a subject can be initiated as a result of a pathogenic infection, such as by a virus, a bacterium, a fungus, a parasite, and the like. Depending on the severity of the infection, many infected subjects need to be placed on ventilators in order to assist them in breathing through their infected lungs.

Nonlimiting examples of respiratory conditions can include chickenpox, coronavirus infections, viral infections, non-viral infections, diphtheria, group A streptococcus, haemophilus influenzae type b, influenza, legionnaires' disease, measles, Middle East Respiratory Syndrome (MERS), mumps, pneumonia, pneumococcal meningitis, rubella, Severe Acute Respiratory Syndrome (SARS), tuberculosis, whooping cough, Acute Respiratory Distress Syndrome (ARDS), and the like.

One nonlimiting example of a pathogenic infection that can produce severe respiratory conditions is the coronavirus disease 2019 (COVID-19), which is caused by a highly contagious coronavirus, SARS-CoV-2. SARS-CoV-2 has rapidly spread around the world since its first detection in Hubei Province, China, in December of 2019. The first patients at the epicenter of the outbreak had a link to a large seafood and live animal market, suggesting an animal-to-person spread. Later waves of patients testing positive for COVID-19 had no association with the original food market, suggesting a person-to-person spread of the disease. It is this mode of disease transmission that has caused the wide and rapid spread of COVID-19 around the world that was declared a pandemic. Severe/critical form of the disease develops in approximately 19% of patients testing positive for SARS-CoV-2. Among these, a mortality rate of 46-49% has been observed. When relying solely on supportive care that consists on mechanical ventilation in support of vital organ functions, development of novel therapies aimed at treatment of patients with severe manifestations of COVID-19 disease, i.e. Acute Lung Injury (ALI) and Acute Respiratory Distress Syndrome (ARDS), who rapidly progress to organ failure, is of critical importance.

This highly contagious coronavirus, SARS-CoV-2, spread rapidly around the world, causing a sharp rise of a pneumonia-like disease termed Coronavirus Disease 2019 (COVID-19). COVID-19 presents a high mortality rate, estimated at 3.4% by the World Health Organization. The rapid spread of the virus (estimated reproductive number RO 2.2 — 3.6) caused a significant surge of patients requiring intensive care. More than 1 out of 4 hospitalized COVID-19 patients have required admission to an Intensive Care Unit (ICU) for respiratory support, and a large proportion of these ICU-COVID-19 patients, between 17% and 46%, have died.

A common observation among patients with severe COVID-19 infection is a hyper-inflammatory response localized to the lower respiratory tract. This inflammation, associated with dyspnea and hypoxemia, in some cases evolves into excessive immune response with cytokine storm, determining progression to Acute Lung Injury (ALI), Acute Respiratory Distress Syndrome (ARDS), organ failure, and death.

Although the majority of subjects infected by SARS-CoV-2 remain asymptomatic or have mild symptoms, a subset develops severe COVID-19 requiring hospitalization. Without intending to be bound to any medical theory, severe COVID-19 is believed to result from hyperinflammation, overactive immune response with cytokine storm, and a pro-thrombotic state elicited by SARS-CoV-2 infection. Subjects progressing to ARDS require high flow oxygen therapy, intensive care, and, frequently, mechanical ventilation.

Mortality in COVID-19 is associated with cytokine storm, ARDS, and multiple organ failure. Mortality in mechanically ventilated patients has been reported to be above 40% at 28 days.

The present disclosure provides novel compositions and therapies to treat respiratory infections, including those leading to ARDS. Such therapies can decrease the hyperinflammatory response, suppress the cytokine storm, and improve survival in patients with severe respiratory infections, ARDS, and the like, including those associated with COVID-19. The present novel therapies can dampen the excessive inflammatory response in the lungs associated with the immunopathological cytokine storm and accelerate the regeneration of functional lung tissue in COVID-19 patients.

The aforementioned therapy includes administering stem cells to a subject obtained from a subepithelial layer of a mammalian umbilical cord tissue that are capable of self-renewal and culture expansion is provided. Such cells are capable of differentiation into a progenitor cell type such as, in one aspect for example, adipocytes, chondrocytes, osteocytes, cardiomyocytes, and the like. In another aspect, non-limiting examples of such cell types can include white, brown, or beige adipocytes, chondrocytes, osteocytes, cardiomyocytes, endothelial cells, myocytes, and the like, including combinations thereof. Other examples of such cell types can include neural progenitor cells, hepatocytes, islet cells, renal progenitor cells, and the like.

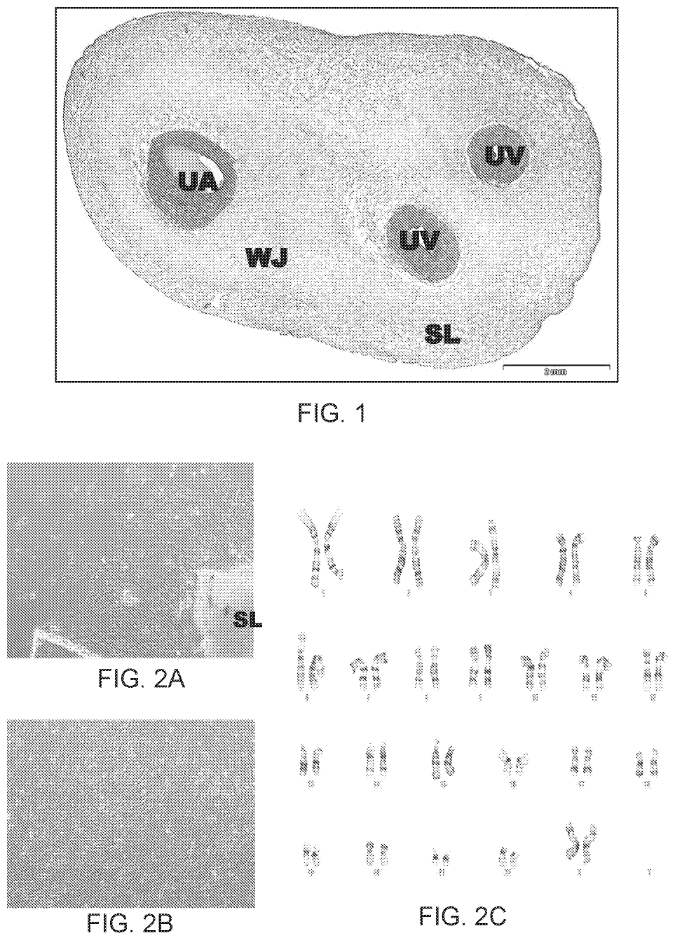

A cross section of a human umbilical cord is shown in FIG. 1, which shows the umbilical artery (UA), the umbilical veins (UV), the Wharton's Jelly (WJ), and the subepithelial layer (SL). Isolated cells from the SL can have a variety of characteristic markers that distinguish them from cell previously isolated from umbilical cord samples. It should be noted that these isolated cells are not derived from the Wharton's Jelly, but rather from the SL.

Various cellular markers that are either present or absent can be utilized in the identification of these SL-derived cells, and as such, can be used to show the novelty of the isolated cells. For example, in one aspect, the isolated cell expresses at least three cell markers selected from CD29, CD73, CD90, CD146, CD166, SSEA4, CD9, CD44, CD146, or CD105, and the isolated cell does not express at least three cell markers selected from CD45, CD34, CD14, CD79, CD106, CD86, CD80, CD19, CD117, Stro-1, or HLA-DR. In another aspect, the isolated cell expresses at least five cell markers selected from CD29, CD73, CD90, CD146, CD166, SSEA4, CD9, CD44, CD146, or CD105. In another aspect, the isolated cell expresses at least eight cell markers selected from CD29, CD73, CD90, CD146, CD166, SSEA4, CD9, CD44, CD146, or CD105. In a yet another aspect, the isolated cell expresses at least CD29, CD73, CD90, CD166, SSEA4, CD9, CD44, CD146, and CD105. In another aspect, the isolated cell does not express at least five cell markers selected from CD45, CD34, CD14, CD79, CD106, CD86, CD80, CD19, CD117, Stro-1, or HLA-DR. In another aspect, the isolated cell does not express at least eight cell markers selected from CD45, CD34, CD14, CD79, CD106, CD86, CD80, CD19, CD117, Stro-1, or HLA-DR. In yet another aspect, the isolated cell does not express at least CD45, CD34, CD14, CD79, CD106, CD86, CD80, CD19, CD117, Stro-1, and HLA-DR. Additionally, in some aspects, the isolated cell can be positive for SOX2, OCT4, or both SOX2 and OCT4. In a further aspect, the isolated cell can produce exosomes expressing CD63, CD9, or both CD63 and CD9.

A variety of techniques can be utilized to extract the isolated cells of the present disclosure from the SL, and any such technique that allows such extraction without significant damage to the cells is considered to be within the present scope. In one aspect, for example, a method of culturing stem cells from the SL of a mammalian umbilical cord can include dissecting the subepithelial layer from the umbilical cord. In one aspect, for example, umbilical cord tissue can be collected and washed to remove blood, Wharton's Jelly, and any other material associated with the SL. For example, in one non-limiting aspect the cord tissue can be washed multiple times in a solution of Phosphate-Buffered Saline (PBS) such as Dulbecco's Phosphate-Buffered Saline (DPBS). In some aspects the PBS can include a platelet lysate (i.e. 10% PRP lysate of platelet lysate). Any remaining Wharton's Jelly or gelatinous portion of the umbilical cord can then be removed and discarded. The remaining umbilical cord tissue (the SL) can then be placed interior side down on a substrate such that an interior side of the SL is in contact with the substrate. An entire dissected umbilical cord with the Wharton's Jelly removed can be placed directly onto the substrate, or the dissected umbilical cord can be cut into smaller sections (e.g. 1-3 mm) and these sections can be placed directly onto the substrate.

The culture can then be cultured under either normoxic or hypoxic culture conditions for a period of time sufficient to establish primary cell cultures. (e.g. 3-7 days in some cases). After primary cell cultures have been established, the SL tissue is removed and discarded. Cells or stem cells are further cultured and expanded in larger culture flasks in either a normoxic or hypoxic culture conditions. While a variety of suitable cell culture media are contemplated, in one non-limiting example the media can be Dulbecco's Modified Eagle Medium (DMEM) glucose (500-6000 mg/mL) without phenol red, 1× glutamine, 1× NEAA, and 0.1-20% PRP lysate or platelet lysate. Another example of suitable media can include a base medium of DMEM low glucose without phenol red, 1X glutamine, 1X NEAA, 1000 units of heparin and 20% PRP lysate or platelet lysate. In another example, cells can be cultured directly onto a semi-solid substrate of DMEM low glucose without phenol red, 1× glutamine, 1× NEAA, and 20% PRP lysate or platelet lysate. In a further example, culture media can include a low glucose medium (500-1000 mg/mL) containing 1× Glutamine, 1× NEAA, 1000 units of heparin. In some aspects, the glucose can be 1000-4000 mg/mL, and in other aspects the glucose can be high glucose at 4000-6000 mg/mL. These media can also include 0.1%-20% PRP lysate or platelet lysate. In yet a further example, the culture medium can be a semi-solid with the substitution of acid-citrate-dextrose ACD in place of heparin, and containing low glucose medium (500-1000 mg/mL), intermediate glucose medium (1000-4000 mg/mL) or high glucose medium (4000-6000 mg/mL), and further containing 133 Glutamine, 1× NEAA, and 0.1%-20% PRP lysate or platelet lysate. In some aspects, the cells can be derived, subcultured, and/or passaged using TrypLE. In another aspect, the cells can be derived, subcultured, and/or passaged without the use of TrypLE or any other enzyme.

A variety of substrates are contemplated upon which the SL can be placed. In one aspect, for example, the substrate can be a solid polymeric material. One example of a solid polymeric material can include a cell culture dish. The cell culture dish can be made of a cell culture treated plastic as is known in the art. In one specific aspect, the SL can be placed upon the substrate of the cell culture dish without any additional pretreatment to the cell culture treated plastic. In another aspect, the substrate can be a semi-solid cell culture substrate. Such a substrate can include, for example, a semi-solid culture medium including an agar or other gelatinous base material.

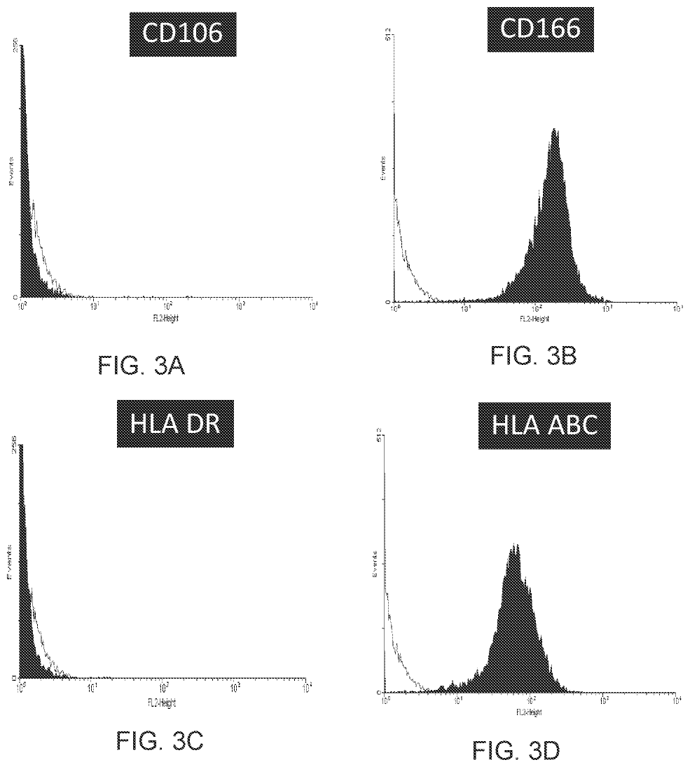

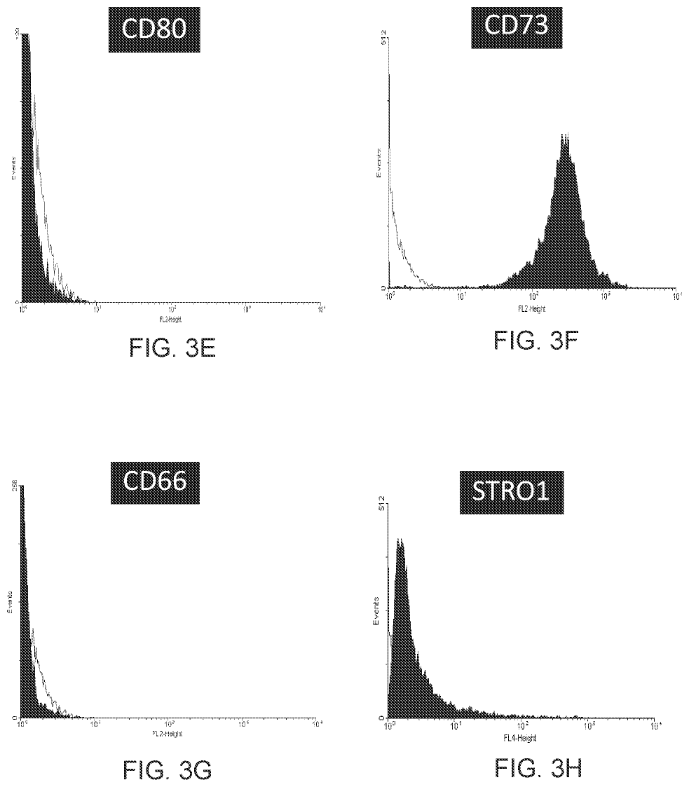

Following placement of the SL on the substrate, the SL is cultured in a suitable medium. In some aspects it is preferable to utilized culture media that is free of animal and human components or contaminants. As one example, FIG. 2 shows the culturing of cells from the SL. As can be seen in FIG. 2A, at three days post plating of the SL, cells have begun to migrate. FIG. 2B shows cells after 6 days of culture in animal free media. Furthermore, FIG. 2C shows the karyotype of cells following passage 12. As has been described, the cells derived from the SL have a unique marker expression profile. Data showing a portion of this profile is shown in FIGS. 3A-O.

One nonlimiting example of a stem cell progenitor useful in the treatments described herein are Mesenchymal Stem Cells (MSCs), also known as Mesenchymal Stromal Cells or Medicinal Signaling Cells, which can modulate overactive immune and hyper-inflammatory processes, promote tissue repair and regeneration, and secrete antimicrobial molecules. These cells may play an important role in the treatment of, without limitation, autoimmune diseases (e.g., type 1 diabetes (T1D) and systemic lupus erythematous), inflammatory disorders, and steroid-refractory Graft-versus-Host-Disease (GvHD) to name a few. MSCs can limit inflammation and fibrosis in the lungs and have generated variable results in ARDS, of viral and non-viral etiology. MSCs can be isolated and expanded from multiple tissues, including the Umbilical Cord (UC). UC-MSC constitute very useful cell type in cell-based therapies, including for COVID-19.

MSCs, and the like, including combinations thereof. Other examples of such cell types can include neural progenitor cells, hepatocytes, islet cells, renal progenitor cells, and the like.

UC-MSC is a safe and effective treatment to prevent the progression of complications associated with the hyper-immune, hyper-inflammatory, and thrombotic responses to such infections, including the SARS-CoV-2 infection in subjects with COVID-19 and ARDS.

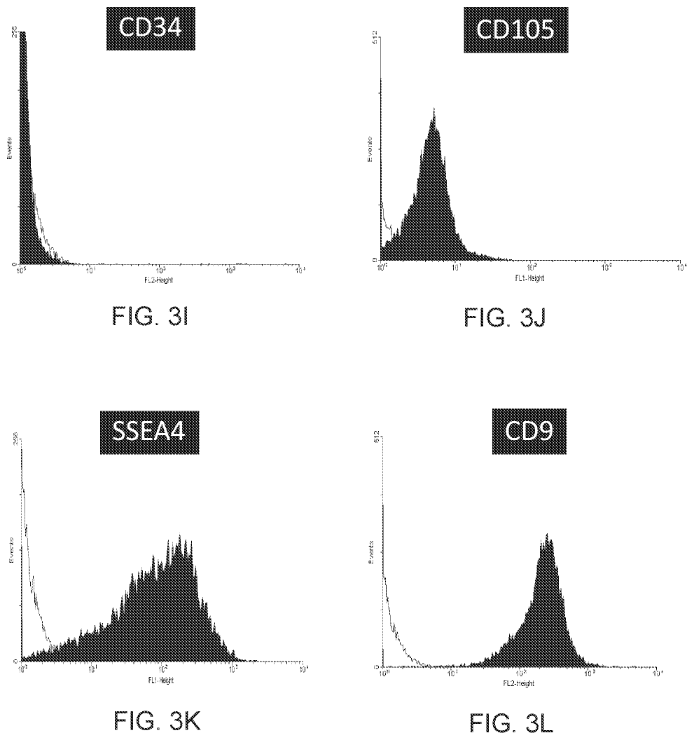

FIG. 4 shows data relating to various genetic characteristics of the cells isolated from the SL tissue. FIG. 4A shows that isolated SL cells (lane 1) are positive for SOX2 and OCT4 and are negative for NANOG as compared to control cells (Ctrl). FIG. 4B shows a DAPI stained image of cultured SL cells demonstrating that such cells are positive for CD44. FIG. 4C shows a DAPI stained image of cultured SL cells demonstrating that such cells are positive for CD90. FIG. 4D shows a DAPI stained image of cultured SL cells demonstrating that such cells are positive for CD146.

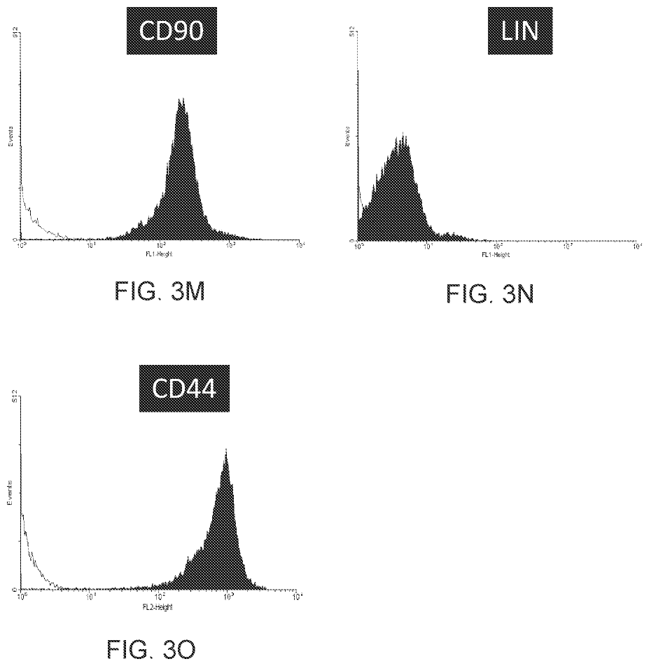

In one aspect, SL cells can be cultured from a mammalian umbilical cord in a semi-solid PRP Lysate or platelet lysate substrate. Such cells can be cultured directly onto a plastic coated tissue culture flask as has been described elsewhere herein. After a sufficient time in either normoxic or hypoxic culture environments the media is changed and freshly made semi-solid PRP lysate or platelet lysate media is added to the culture flask. The flask is continued to be cultured in either a normoxic or hypoxic culture environment. The following day the media becomes a semi-solid PRP-lysate or platelet lysate matrix. The cells can be continued to be cultured in this matrix being until further use. FIGS. 5A and B show SL cells growing in a semi-solid PRPL or PL gel at 10× and 40× magnifications. In one specific aspect, ingredients for a semi solid culture can include growth factors for expanded cell culture of differentiation. Non-limiting examples can include FGF, VEGF, FNDC5, 5-azacytidine, TGF-Betal, TGF Beta2, insulin, ITS, IGF, and the like, including combinations thereof.

In some cases, allogenic confirmation of SL cells, either differentiated or undifferentiated, can be highly beneficial, particularly for therapeutic uses of the cells. In such cases, mixed lymphocyte reactions can be performed on the cells to confirm the allogenic properties of the cells. In certain aspects, a cell derived as described herein does not cause a mixed lymphocyte response or T-cell proliferation.

In certain aspects, a cell derived as described herein can be recombinantly modified to express one or more genes and or proteins. In one technique, a gene or genes can be incorporated into an expression vector. Approaches to deliver a gene into the cell can include without limitation, viral vectors, including recombinant retroviruses, adenoviruses, adeno-associated virus, lentivirus, poxivirus, alphavirus, herpes simplex virus-1, recombinant bacterial, eukaryotic plasmids, and the like, including combinations thereof. Plasmid DNA may be delivered naked or with the help of exosomes, cationic liposomes or derivatized (antibody conjugated) polylysine conjugates, gramicidin S, artificial viral envelopes, other intracellular carriers, as well as direct injection of the genes. In some aspects, non-viral gene delivery methods can be used, such as for example, scaffold/matrix attached region (S/MAR)-based vector.

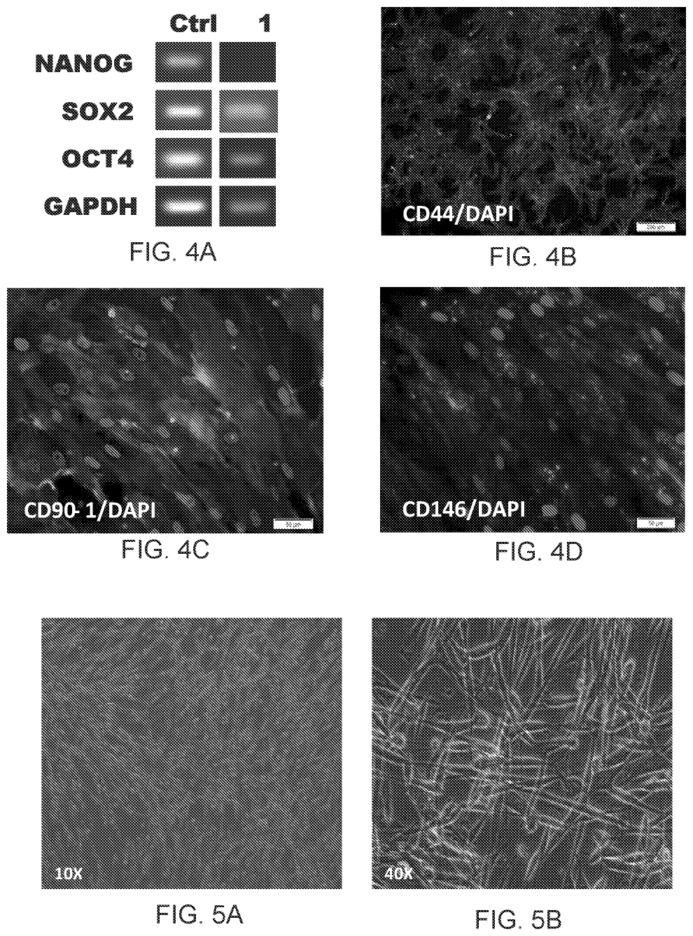

Furthermore, in some aspects, isolated SL cells can be used to produce an exosome population. These exosome populations can be utilized for a variety of research and therapeutic uses. In one aspect, for example, cells are cultured as described in either a normoxic or hypoxic culture environment and supernatants are collected at each media change. Exosomes can then be purified from the supernatants using an appropriate purification protocol. One not-limiting example of such a protocol is the ExoQuick isolation system by SYSTEMBIO. Purified exosomes can be utilized for further manipulation, targeting, and therapeutic use. The exosomes specific to the SL cells are positive for CD63 expression. FIG. 6A shows an analysis of the size of exosomes obtained as has been described, and FIG. 6B shows and electron microscope image of a sampling of exosomes. Additionally, FIGS. 6C-D show CD63 expression of exosomes produced from cells or stem cells derived from umbilical cord.

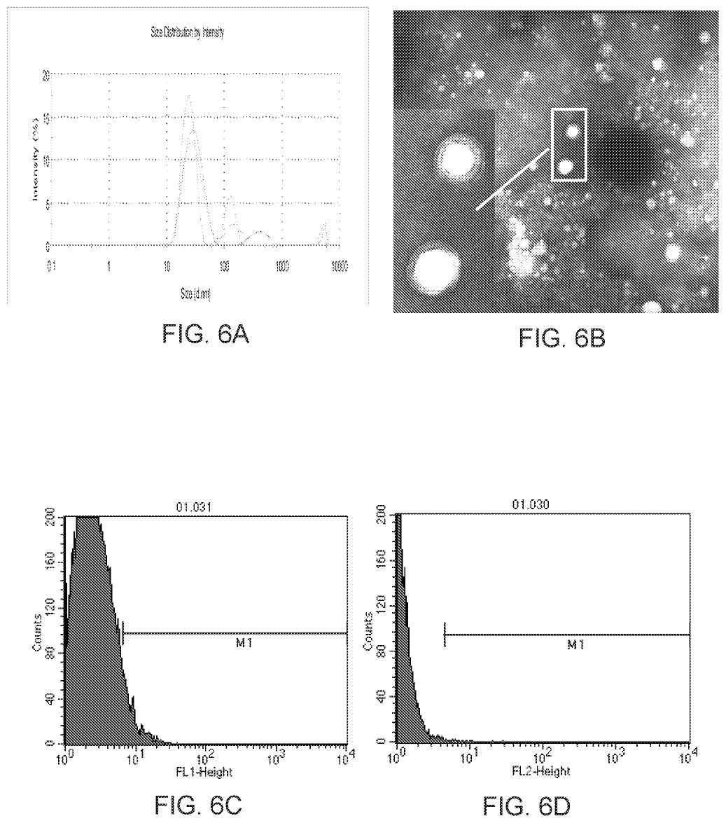

In some aspects, the isolated cells and cell cultures can be utilized as-is upon isolation from the SL tissue. In other aspects, the isolated cells can be differentiated into progenitor cells other cell types. It should be noted that any useful cell type that can be derived from the cells isolated from SL tissue are considered to be within the present scope. Non-limiting examples of such cell types include adipocytes, chondrocytes, osteocytes, cardiomyocytes, and the like. Differentiation can be induced by exposing the cells to chemicals, growth factors, supernatants, synthetic or naturally occurring compounds, or any other agent capable of transforming the cells. In one aspect, for example, the isolated cells can be differentiated into adipocytes, as is shown in FIG. 7.

Any technique for differentiation of SL cells into adipocytes is considered to be within the present scope. One non-limiting example used for adipogenic differentiation includes SL cells cultured in the presence of StemPro Adipogenic Differentiation media (Life Technologies). FIG. 7A shows differentiated SL cells that are positive for the adipogenic markers FABP4, LPL, and PPARy (lane 1). For adipogenic differentiation, confirmation was determined by Oil Red O staining and FABP4 immunocytochemistry. FIG. 7B shows an image of DAPI stained cells showing FABP4 markers. FIG. 7C shows unstained cells and FIG. 7D shows Oil Red O staining demonstrating the storage of fats in the cells.

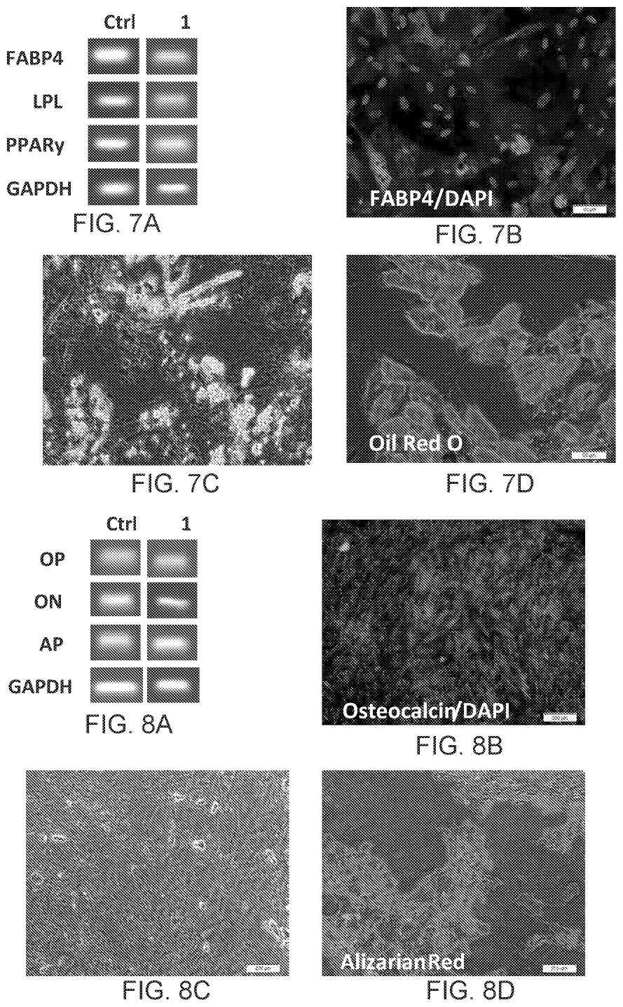

For osteogenic differentiation of SL cells, one non-limiting technique cultures such cells in the presence of StemPro Osteogenic Differentiation media (Life Technologies). As is shown in FIG. 8A, for example, differentiated SL cells are positive for the osteogenic markers OP, ON, and AP (lane 1). For osteogenic differentiation, confirmation was determined by Alizarin red staining and osteocalcin immunocytochemistry. FIG. 8B shows an image of DAPI stained cells showing the presence of osteocalcin. FIG. 8C shows unstained cells and FIG. 8D shows an image of cells stained with alizarin red demonstrating the presence of calcific deposition in the cells.

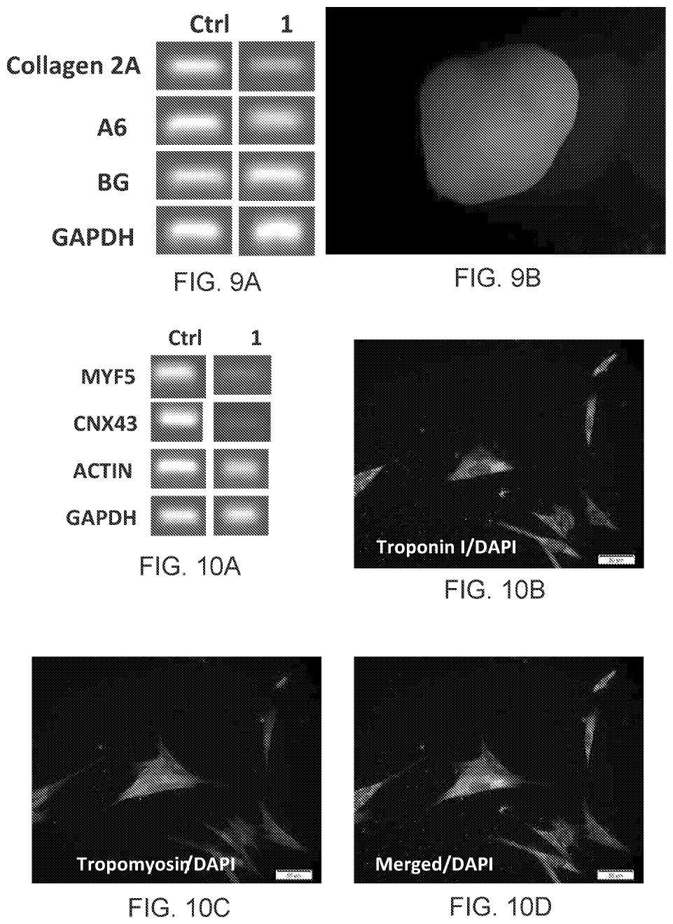

For chondrogenic differentiation of SL cells, one non-limiting technique cultures SL cells in the presence of StemPro Chondrogenic Differentiations media (Life Technologies). As is shown in FIG. 9A, differentiated SL cells are positive for chondrogenic markers Collagen 2A, A6, and BG (lane 1). For chondrogenic differentiation, confirmation was determined by Von Kossa staining. FIG. 9B shows Alcian blue staining of a chondrocyte pellet.

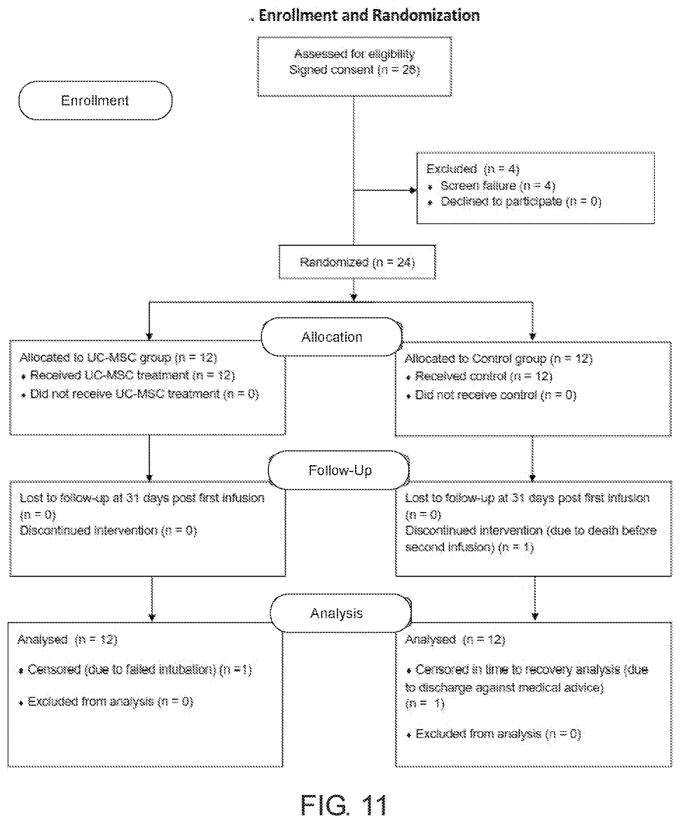

For cardiogenic differentiation of SL cells, one non-limiting technique cultures cells in the presence of DMEM low glucose without phenol red, 1× glutamine, 1× NEAA and 10% PRP lysate or platelet lysate with 5-10 μM 5-AZA-2′-deoxycytidine. As is shown in FIG. 10A, differentiated SL cells are positive for the cardiogenic markers MYFS, CNX43, and ACTIN (lane 1). For cardiogenic differentiation, confirmation was determined by staining for ANP, tropomyosin, and troponin 1. FIG. 10B shows an image of DAPI stained cells demonstrating the presence of Troponin 1. FIG. 10C shows an image of DAPI stained cells demonstrating the presence of tropomyosin. FIG. 10D shows a merged image of the images from FIGS. 10B and 10C.

In yet another aspect, a method of treating a medical condition is provided. In some embodiments, such a method can include introducing cells described herein into an individual having the medical condition. Cells can be delivered at various doses such as, without limitation, from about 500,000 to about 1,000,000,000 cells per dose. In some aspects, the cell dosage range can be calculated based on the subject's weight. In certain aspects, the cell range is calculated based on the therapeutic use or target tissue or method of delivery.

Stem cells can also be delivered into an individual according to retrograde or antegrade delivery. As an example, cells can be introduced into an organ of an individual via retrograde delivery of the cells into the organ. Non-limiting examples of such organs can include the heart, the liver, a kidney, the brain, pancreas, and the like.

Additionally, in some aspects SL cells can be lysed and the lysate used for treatment. In other aspects, supernatant from the culture process can be used for treatment. One example of such a supernatant treatment includes the delivery of exosomes. Exosomes can be delivered into the individual via aerosolized delivery, IV delivery, or any other effective delivery technique.

A variety of conditions can be utilized to aerosolize cells. In one aspect, for example, cells can be suspended in 1-5 mls of saline and aerosolized at a pressure of 3-100 psi for 1-15 minutes, or until the cells begin to rupture and/or die.

Any form of aerosolizer can be utilized to deliver stem cells to the lungs provided the stem cells can be delivered substantially without damage. In some cases, it can be beneficial to aerosol stem cells via an aerosolizer capable of aerosolizing particles to larger sizes. For example, in one aspect, an aerosolizer can be used that aerosolizes to a particle size of from about 2 microns to about 50 microns. In another aspect, an aerosolizer can be used that aerosolizes to a particle size of from about 4 microns to about 30 microns. In yet another aspect, an aerosolizer can be used that aerosolizes to a particle size of from about 6 microns to about 20 microns. In yet another aspect, an aerosolizer can be used that aerosolizes to a particle size of from about 6 microns to about 200 microns.

### EXAMPLES

### Methods: Clinical Trial Design

The following is an early phase 1/2a, double-blind randomized controlled trial (RCT) performed at the UHealth System/Jackson Health System. The trial is designed to evaluate safety and explore efficacy endpoints of allogeneic UC-MSC in COVID-19 patients with ARDS. Twenty-four subjects hospitalized for COVID-19 and confirmed positive for SARS-CoV-2 by PCR, were randomized 1:1 to either UC-MSC treatment group (n=12; Group A) or Control group (n=12; Group B). Subjects in the UC-MSC treatment group received two intravenous infusions (IV) of 100±20×10⁶ UC-MSC each, in vehicle solution containing Human Serum Albumin (HSA) and heparin. The first infusion was administered within 24 hours from randomization (day 0), with the second infusion administered at 72±6 hours thereafter. Subjects in the Control group (n=12) received two infusions of vehicle solution containing HSA and heparin (Placebo), 72 hours apart. Best standard of care was provided in both groups. Fifty ml of UC-MSC or vehicle solution were infused intravenously (IV) over 10±5 min. Infusion-associated Adverse Events (AEs) and Serious Adverse Events (SAEs) were monitored in all subjects for 6 hours. AEs and SAEs were assessed for 31 days following treatment.

### Patient Recruitment and Enrollment

The U.S. FDA authorized the study to proceed and the protocol received IRB approval prior to the first subject being enrolled. Patients were pre-screened for enrollment based on meeting eligibility criteria by pulmonary intensivists at UHealth System/Jackson Health System in Miami, Florida (US). The research team confirmed eligibility based on clinical parameters for enrollment. Informed consent was obtained from the patient or by proxy, depending on subject's health status and oxygen-support requirements. The PaO₂/FiO₂ ratio was utilized for severity stratification at the time of randomization.

Subjects diagnosed with COVID-19 were eligible for inclusion if they met the following criteria within 24-hour time period of enrollment: patient currently hospitalized; aged ≥18 years; peripheral capillary oxygen saturation (SpO₂)≤94% at room air, or requiring supplemental oxygen at screening; PaO₂/FiO₂ ratio<300 mmHg; bilateral infiltrates on frontal chest radiograph or bilateral ground glass opacities on a chest CT scan. Eligibility criteria, including inclusion and exclusion criteria, are listed in Table 1.

TABLE 1
Eligibility Criteria

Inclusion Criteria
Patient currently hospitalized
Aged ≥18 years
Willing and able to provide written informed consent, or with a legal representative who can provide informed consent
Peripheral capillary oxygen saturation (SpO2) ≤94% at room air, or requiring supplemental oxygen at screening
PaO2/FiO2 ratio <300 mmHg
Bilateral infiltrates on frontal chest radiograph or bilateral ground glass opacities on a chest CT scan
Exclusion Criteria
PaO₂/FiO₂ ≥300 at the time of enrollment
A previous MSC infusion not related to this trial
History of Pulmonary Hypertension (WHO Class III/IV)
History of left atrial hypertension or decompensated left heart failure.
Pregnant or lactating patient
Unstable arrhythmia
Patients with previous lung transplant
Patients currently receiving chronic dialysis
Patients currently receiving Extracorporeal Membrane Oxygenation (ECMO)
Presence of any active malignancy (except non-melanoma skin cancer)
Any other irreversible disease or condition for which 6-month mortality is estimated to be greater than 50%
Moderate to severe liver disease (AST and ALT >5 × ULN)
Severe chronic respiratory disease with a PaCO₂ >50 mm Hg or the use of home oxygen
Baseline QT prolongation
Moribund patient not expected to survive >24 hours

### Outcome Measures

Primary Endpoints of this trial:
1. Safety, as defined by the occurrence of pre-specified infusion associated AEs, occurring within 6 hours from each infusion:
   a. An increase in vasopressor dose greater than or equal to the following:
      i. Norepinephrine: 10 μg per min
      ii. Phenylephrine: 100 μg per min
      iii. Dopamine: 10 μg/kg per min
      iv. Epinephrine: 10 μg per min
   b. In patients receiving mechanical ventilation: worsening hypoxemia, as assessed by a requirement for an increase of PEEP by 5 cm H₂O over baseline, or requirement of a percentage increase in FiO₂ of >20% from baseline
   c. In patients receiving high flow oxygen therapy: worsening hypoxemia, as indicated by requirement of intubation and mechanical ventilation
   d. New cardiac arrhythmia requiring cardioversion
   e. New ventricular tachycardia, ventricular fibrillation, or asystole
   f. A clinical scenario consistent with transfusion incompatibility or transfusion-related infection
2. Cardiac arrest or death within 24 h post infusion
3. Incidence of AEs

Secondary Endpoints included exploratory efficacy defined by
1. Survival at day 28
2. Time to recovery

### Statistical Design and Analytical Methods

Patients were assigned to treatment group using a stratified, blocked randomized design. Stratification was determined by ARDS severity, defined by a ratio of the partial pressure of arterial oxygen to the fraction of inspired oxygen (PaO₂/FiO₂), with mild-to-moderate ARDS defined as a ratio >150 mm Hg and moderate-to-severe ARDS defined as a ratio ≤150 mm Hg. Blocking in the randomization scheme was implemented to ensure balance among treatment groups in standard of care practices over time, thus any changes in standard of care over time would be reflected evenly between treatment groups.

Concurrent parallel controls were utilized to estimate differences in Adverse Events (AEs), SAES, and clinical indicators. Safety and efficacy analysis were performed by trained staff at trial sites and by the authors, with the oversight from the PI, study sponsor. There were no major protocol deviations affecting primary and secondary end points.

### Two Cases Required Censoring of Data

Subject #11 died after failed endotracheal intubation. Death was deemed to be unrelated to the patient's course of COVID-19 related illness. This subject was censored in the data analysis for time to death and time to recovery outcomes.

Subject #24 left the hospital 11 days after second infusion against medical advice and was thus considered censored in the analysis for time to recovery. This patient was confirmed alive at 31 days post first infusion.

### Investigational Product: UC-MSC

Allogeneic UC-MSC for this clinical trial were derived from a single, previously established and characterized Master Cell Bank (MCB) prepared from a single healthy donor (kindly provided by Jadi Cell and Amit Patel, MD). The MCB and its source tissue were tested according to the applicable FDA regulations and AABB and FACT standards for cellular therapies. UC-MSC were manufactured as previously described.

In preparation for infusion, UC-MSCs were removed from cold storage, quickly thawed and slowly diluted in Plasma-Lyte supplemented with HSA and Heparin (vehicle solution). The final volume of UC-MSC suspension or vehicle solution (control) for infusion was 50 ml. Cell dose (100±20×10⁶), cell viability by Trypan Blue (>80%), cell surface marker expression by flow cytometric analysis (CD90/CD105 >95%, CD34/CD45 <5%), Endotoxin, <1.65 EU/ml), Gram stain (negative) and 14-Day Sterility (negative) were utilized as product release criteria. UC-MSCs were also assessed for viability (fixable viability stain) and apoptosis (activated Caspase-3) by flow cytometry. Vehicle solution was tested for 14-day sterility, Gram stain and Endotoxin. The UC-MSC suspension or vehicle solution was infused within 3 hours of preparation for infusion.

### Results

### Clinical Trial Participants

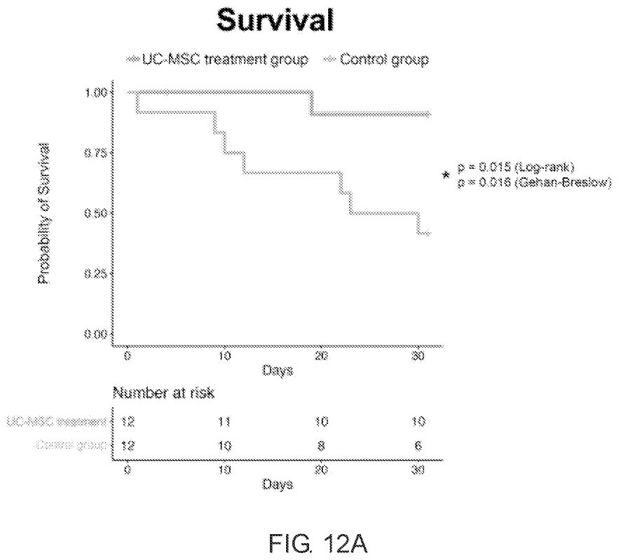

From April 25 to Jul. 21, 2020, the study enrolled 28 subjects (See FIG. 11, 102). Four subjects were subsequently determined to be ineligible due to screen failure (104). The study successfully randomized 24 subjects (106). At enrollment, n=11 subjects (46%) were receiving invasive mechanical ventilation, and n=13 (54%) on high flow oxygen therapy via non-invasive ventilation [including High Flow Nasal Cannula (HFNC), continuous positive airways pressure (CPAP), or bilevel positive airways pressure (BiPAP)] prior to initiation of treatment.

Demographics and baseline characteristics for enrolled subjects, along with stratification and randomization information, are presented in Table 2 and Table 3.

TABLE 2
Demographics, Stratification, and Randomization Information

| | | | | | | | PaO₂/FiO₂ | | |
|---|---|---|---|---|---|---|---|---|---|
| | | | Race | | Ethnicity | | Ratio at | ARDS Severity Stratification | |
| | | Female/Male | | African | Hispanic | Non- | Enrollment ± | Mild-to- | Moderate-to- |
| Treatment | Age ± SD | Ratio | White | American | or Latino | Hispanic | SD | Moderate | Severe |
| UC-MSC | 59 ± 16 | 7/5 | 11 | 1 | 11 | 1 | 127 ± 64 | 3 | 9 |
| Control | 58 ± 11 | 4/8 | 10 | 2 | 11 | 1 | 118 ± 63 | 3 | 9 |
| All Subjects | 58 ± 14 | 11/13 | 21 | 3 | 22 | 2 | 122 ± 63 | 6 | 18 |

TABLE 3
Demographic, Stratification, and Randomization Information

| | | | | | | PaO₂/FiO₂ | |
|---|---|---|---|---|---|---|---|
| | | | | | | Ratio at | ARDS Severity |
| Treatment | Subject # | Age | Gender | Race | Ethnicity | Enrollment | Stratification |
| UC-MSC | 1 | 49 | Female | White | Hispanic | 191 | Mild-to-Moderate |
| | | | | | or Latino | | |
| UC-MSC | 4 | 55 | Female | African | Non-Hispanic | 223 | Mild-to-Moderate |
| | | | | American | | | |
| UC-MSC | 6 | 73 | Female | White | Hispanic | 135 | Moderate-to-Severe |
| | | | | | or Latino | | |
| UC-MSC | 7 | 72 | Female | White | Hispanic | 54 | Moderate-to-Severe |
| | | | | | or Latino | | |
| UC-MSC | 10 | 33 | Male | White | Hispanic | 73 | Moderate-to-Severe |
| | | | | | or Latino | | |
| UC-MSC | 11 | 64 | Female | White | Hispanic | 113 | Moderate-to-Severe |
| | | | | | or Latino | | |
| UC-MSC | 14 | 86 | Female | White | Hispanic | 63 | Moderate-to-Severe |
| | | | | | or Latino | | |
| UC-MSC | 15 | 65 | Male | White | Hispanic | 99 | Moderate-to-Severe |
| | | | | | or Latino | | |
| UC-MSC | 16 | 30 | Male | White | Hispanic | 251 | Mild-to-Moderate |
| | | | | | or Latino | | |
| UC-MSC | 20 | 59 | Female | White | Hispanic | 137 | Moderate-to-Severe |
| | | | | | or Latino | | |
| UC-MSC | 21 | 60 | Male | White | Hispanic | 136 | Moderate-to-Severe |
| | | | | | or Latino | | |
| UC-MSC | 23 | 57 | Male | White | Hispanic | 46 | Moderate-to-Severe |
| | | | | | or Latino | | |
| Control | 2 | 80 | Male | African | Hispanic | 199 | Mild-to-Moderate |
| | | | | American | or Latino | | |
| Control | 3 | 68 | Female | White | Hispanic | 235 | Mild-to-Moderate |
| | | | | | or Latino | | |
| Control | 5 | 42 | Female | White | Hispanic | 115 | Moderate-to-Severe |
| | | | | | or Latino | | |
| Control | 8 | 54 | Male | White | Hispanic | 132 | Moderate-to-Severe |
| | | | | | or Latino | | |
| Control | 9 | 58 | Male | White | Hispanic | 61 | Moderate-to-Severe |
| | | | | | or Latino | | |
| Control | 12 | 49 | Male | White | Hispanic | 76 | Moderate-to-Severe |
| | | | | | or Latino | | |
| Control | 13 | 68 | Male | White | Hispanic | 108 | Moderate-to-Severe |
| | | | | | or Latino | | |
| Control | 17 | 65 | Female | White | Hispanic | 213 | Mild-to-Moderate |
| | | | | | or Latino | | |
| Control | 18 | 70 | Female | White | Hispanic | 37 | Moderate-to-Severe |
| | | | | | or Latino | | |
| Control | 19 | 44 | Male | White | Hispanic | 51 | Moderate-to-Severe |
| | | | | | or Latino | | |
| Control | 22 | 58 | Male | White | Hispanic | 77 | Moderate-to-Severe |
| | | | | | or Latino | | |
| Control | 24 | 50 | Male | African | Non-Hispanic | 109 | Moderate-to-Severe |
| | | | | American | | | |

Twenty-four subjects were randomized: n=12 in the UC-MSC treatment group and n=12 in the Control group. The mean age of enrolled subjects was 58±14 (mean±SD). The average age was 59±16 in the UC-MSC treatment group and 58±11 in the Control group. Among randomized subjects, the Female/Male ratio was 11/13. The Female/Male ratio was 7/5 in the UC-MSC treatment group, and 4/8 in the Control group. At study entry, 3 subjects in each group were in the mild-to-moderate ARDS severity stratum and 9 subjects in each group were in the moderate-to-severe stratum. (See Table 2, and Table 3).

### Investigational Product: UC-MSC

An average of 98.7×10⁶ UC-MSC were administered per infusion. The viability of UC-MSC (Investigational product) at the time of product release for administration was found to range from 91.4% to 99.8% (96.2±1.8%) by Trypan Blue and from 66.82% to 93.8% (88.4±7.7%) by flow cytometry using fixable viability stain. Apoptosis, assessed by activated Caspase-3, was found to be 2.4±3.7%, by flow cytometry. No differences in cell dose, cell viability or degree of apoptosis were observed between UC-MSC (Investigational product) prepared for the first or second infusion. Our stability studies demonstrated stability of the UC-MSC Investigational product for up to 8 hours following thawing and preparation, as assessed by cell count, viability by Trypan Blue and flow cytometry, and apoptosis assessed by flow cytometry.

### Follow-up and Clinical Outcomes

At the date of this writing, all of the patients have been followed for 31 days post first infusion, corresponding to 28 days post second infusion.

### Deaths

Out of 24 patients randomized, 9 deaths occurred by day 28 post second infusion. Seven deaths occurred in the Control group and 2 deaths occurred in the UC-MSC treatment group:

One subject (Subject #11) died after failed endotracheal intubation. Death was deemed to be unrelated to the patient's course of COVID-19 related illness. This subject was censored in the data analysis at the time of failed endotracheal intubation. The details of all deaths are presented in Table 4.

TABLE 4
Deaths

| | | Investigator's | |
|---|---|---|---|
| Subject # | Death | Event Attribution | Treatment |
| 2 | Death secondary to acute respiratory | Unrelated | Control |
| | failure. | | |
| 5 | Death secondary to multi-organ | Unrelated | Control |
| | dysfunction syndrome. | | |
| 9 | Death secondary to multi-organ | Unrelated | Control |
| | dysfunction syndrome. | | |
| 11 | Death secondary to failed endotracheal | Unrelated | UC-MSC |
| | intubation. | | |
| 12 | Death secondary to multi-organ | Unrelated | Control |
| | dysfunction syndrome. | | |
| 14 | Death secondary to acute respiratory | Unrelated | UC-MSC |
| | failure. | | |
| 18 | Death secondary to multi-organ | Unrelated | Control |
| | dysfunction syndrome. | | |
| 19 | Death secondary to multi-organ | Unrelated | Control |
| | dysfunction syndrome. | | |
| 22 | Death secondary to multi-organ | Unrelated | Control |
| | dysfunction syndrome. | | |

### Adverse Events

Two Serious Adverse Events (SAEs) were observed in the UC-MSC group and 16 SAEs in the Control group, affecting 2 vs 8 subjects, respectively (p=0.04 Fisher's exact test). There were significantly more subjects experiencing SAEs in the Control group than in the UC-MSC treatment group. The adverse events in all subjects are summarized in Table 5.

TABLE 5
Summary of All Adverse Events for Randomized Subjects
Table 2. Summary of All Adverse Events for Randomized Subjects

| | | | | | Total | | |
|---|---|---|---|---|---|---|---|
| | UC-MSC | | | | N = 24 (12 | | Fisher's |
| Topics | Treatment | | Controls | | per group) | | exact test |
| Number of AEs reported | 35 | | 53 | | 88 | | |
| Number of Subjects with AEs [1] | 8 | | 11 | | 19 | | NS |
| Number of SAEs reported | 2 | | 16 | | 18 | | |
| Number of Subjects with SAEs [1] | 2 | | 8 | | 10 | | p = 0.04 |
| Number of AEs by Severity* | | | | | | | |
| Mild | 13 | (37%) | 13 | (24%) | 26 | (30%) | |
| Moderate | 18 | (51%) | 21 | (40%) | 39 | (44%) | |
| Severe | 4 | (12%) | 19 | (36%) | 23 | (26%) | |
| Subjects with AEs by Severity [2]** | | | | | | | |
| Mild | 7 | (44%) | 5 | (25%) | 12 | (33%) | NS |
| Moderate | 7 | (44%) | 8 | (40%) | 15 | (42%) | NS |
| Severe | 2 | (12%) | 7 | (35%) | 9 | (25%) | NS |
| Number of AEs by Relatedness to | | | | | | | |
| Treatment* | | | | | | | |
| Unrelated | 31 | (89%) | 45 | (85%) | 76 | (86%) | |
| Unlikely | 3 | (9%) | 7 | (13%) | 10 | (11%) | |
| Possible | 1 | (3%) | 1 | (2%) | 2 | (3%) | |
| Probable | 0 | (0%) | 0 | (0%) | 0 | (0%) | |
| Definite | 0 | (0%) | 0 | (0%) | 0 | (0%) | |
| Subjects with AEs by Relatedness to | | | | | | | |
| Treatment [2]** | | | | | | | |
| Unrelated | 8 | (80%) | 10 | (67%) | 17 | (71%) | NS |
| Unlikely | 1 | (10%) | 4 | (26%) | 5 | (21%) | NS |
| Possible | 1 | (10%) | 1 | (7%) | 2 | (8%) | NS |
| Probable | 0 | (0.0%) | 0 | (0.0%) | 0 | (0.0%) | NS |
| Definite | 0 | (0.0%) | 0 | (0.0%) | 0 | (0.0%) | NS |

[1] Subjects who experience one or more AEs or SAEs are counted only once.
[2] Subjects are counted only once within a particular severity grade or relatedness category.
*Percentages are based on number of AEs reported for each treatment group.
**Percentages are based on N for each treatment group.

### Outcomes Data

### Primary Endpoint

The primary endpoint was safety, as defined by the occurrence of pre-specified infusion associated AEs within 6 h, cardiac arrest or death within 24 h post infusion. UC-MSC treatment was found to be safe, as it did not lead to an increase in pre-specified infusion associated AEs. In the UC-MSC treatment group, the only adverse event occurred in a bradycardic subject, who experienced worsening of bradycardia and required transient vasopressor treatment. In the Control group, all 5 pre-specified infusion associated AEs occurred in the same subject. All pre-specified infusion associated AEs are outlined in Table 6.

TABLE 6
Primary Endpoint Safety
Safety: as defined by the occurrence of pre-specified infusion-associated
adverse events within 6 hours (1.a-1.f) and occurrence of cardiac arrest
or death within 24 h post infusion (2).

| | Adverse Events | |
|---|---|---|
| | UC-MSC | |
| | Treatment | Control |
| | (n = 12) | (n = 12) |
| 1.a An increase in vasopressor dose. | 1 | 1 |
| 1.b In patients receiving mechanical | 0 | 0 |
| ventilation: worsening hypoxemia | | |
| 1.c In patients receiving high flow oxygen | 0 | 0\| |
| therapy: worsening hypoxemia, as indicated | | |
| by requirement of intubation and mechanical | | |
| ventilation | | |
| 1.d New cardiac arrhythmia requiring | 0 | 1 |
| cardioversion | | |
| 1.e New ventricular tachycardia, ventricular | 0 | 1 |
| fibrillation, or asystole | | |
| 1.f A clinical scenario consistent with | 0 | 0 |
| transfusion incompatibility or transfusion- | | |
| related infection | | |
| 2. Cardiac arrest or death within 24 h post | 0 | (1)(29 hrs.) |
| infusion | | |

### Secondary Endpoints

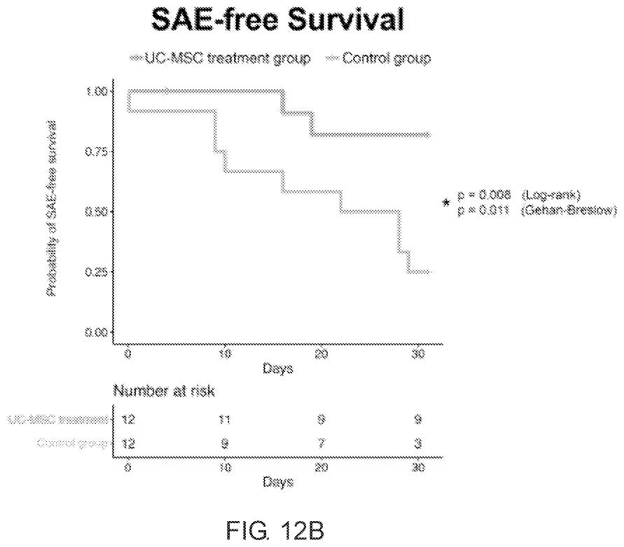

At 31 days post first infusion (corresponding to 28 days post last infusion), patient survival was 91% vs. 42% in the UC-MSC and Control group, respectively (p=0.015). The difference between the groups resulted statistically significant; Kaplan-Meier estimates are presented in FIG. 12A - Survival. SAE-free survival was significantly improved in the UC-MSC treatment group compared to the Control group (p=0.0081). Kaplan-Meier estimates are presented in FIG. 12B—SAE-free survival. Time to recovery was significantly shorter in the UC-MSC treatment group compared to the Control group (p=0.0307). Kaplan-Meier estimates are presented in FIG. 12C—Time to recovery.

### DISCUSSION

Most patients with COVID-19 have mild-to-moderate symptoms involving the upper respiratory system. However, in more severe cases patients can develop ARDS leading to a worse prognosis. Severe COVID-19 is believed to be the result of an hyperinflammatory state and overactive immune response with cytokine storm elicited by SARS-CoV-2 infection. Although the recent RECOVERY trial supports the use of dexamethasone in more severe COVID-19 patients with acute respiratory failure, there is an urgent need for therapies that can dampen the excessive inflammatory response and further improve survival. Mortality in COVID-19 is associated with cytokine storm, ARDS and multiple organ failure, estimated to be above 40% at 28 days in mechanically ventilated patients.

UC-MSC may have safe and beneficial effects in patients with severe COVID-19, based on their abilities to alter the immunopathogenic cytokine storm. These cells, derived from the Umbilical Cord (UC), can be rapidly expanded for clinical applications. UC-MSC were reported to be safe in clinical trials in other disease states and have been safely administered across histocompatibility barriers. Clinical applications utilizing UC-MSCs processed at our cGMP facility have been authorized by the FDA in subjects with T1D (IND#018302) and Alzheimer's Disease (IND#18200). As COVID-19 reached pandemic proportions, it was felt that UC-MSC could also exert beneficial therapeutic effects in subjects with COVID-19 with ARDS. The purpose of this RCT was to determine safety and explore efficacy of UC-MSC for treatment of subjects with COVID-19 and ARDS.

The current report presents, for the first time, the results of a double-blind phase1/2a randomized controlled trial of UC-MSC in 24 subjects with COVID-19 and ARDS. No serious adverse events related to UC-MSC infusion were observed. At 28 days post last infusion, patient survival was 91% vs. 42% in the UC-MSC and Control group, respectively (p=0.015). Two SAEs were reported in the UC-MSC group and 16 in the Control group, affecting 2 vs. 8 patients, respectively (p=0.04). SAE-free survival (p=0.008) and time to recovery (p=0.03) were significantly improved in the UC-MSC treatment group compared to the Control group.

The results of this trial indicated that UC-MSC infusions in COVID-19 with ARDS were safe. Moreover, UC-MSC treatment was associated with a reduction in SAEs, mortality, and time to recovery, compared to controls. These results may be of assistance in the design of expanded Phase 2b/3 RCT to further assess the efficacy of UC-MSC in COVID-19. In addition, synergistic combination strategies could be explored, with agents that show beneficial effects at different stages of COVID-19 disease progression—such as remdesivir and dexamethasone.

The study was originally designed to evaluate safety and early signs of efficacy (Phase 1/2a) and stratified by ARDS severity only, which gives rise to potential imbalance among other prognostic factors. Strikingly, even with a small number of randomized subjects (n=24), significant efficacy outcomes have been observed. These observations should pave the way for regulatory agencies to consider expanded access protocols and larger randomized clinical trials, to confirm efficacy in a larger, geographically and ethnically diverse population, in the immediate future.

The results of this trial indicated that UC-MSC infusions in COVID-19 with ARDS were safe, were associated with fewer SAEs and with a reduction in both mortality and time to recovery, compared to controls.

### Potency Assay

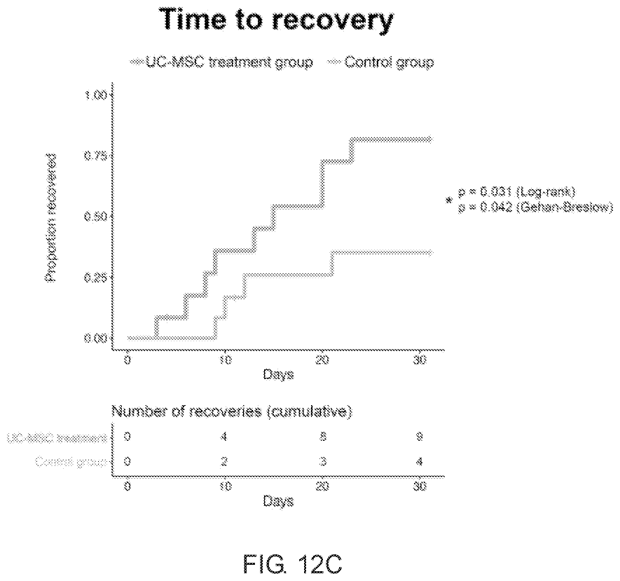

A qualified a biologically relevant potency assay is presented. The potency assay is focused on the measurement of soluble tumor necrosis factor 2 (sTNFR2) release by Umbilical Cord-derived Mesenchymal Stem Cells (UC-MSC). It is based on sTNFR2 quantification via ELISA, normalized based on total cell protein content, and calculation of the Inflammatory Stimulation Index (ISI) of sTNFR2 released by UC-MSC. The ISI is calculated as the ratio of sTNFR2 release in inflammatory induction over basal condition. The assay is performed with in vitro cultures of thawed UC-MSC from the stage of “UC-MSC Final Product (Batch, Cryopreserved)”. The basal condition corresponds to culturing the cells in the same medium utilized for the generation of the final cell product, as described in the CMC section. The inflammatory induction derives from addition of TNFα (15 ng/mL) and IFNγ (10 ng/mL) in the medium of these cultures. The cells are maintained in basal conditions or under inflammatory induction for 3 days of culture. The supernatant is then collected and tested with a commercially available kit for sTNFR2 quantification (Abcam Soluble TNFR2 Human ELISA KIT, Cat # ab100643). For normalization based on total cell protein content, the cells are lysed with RIPA buffer (ThermoFisher Scientific, Cat # 89900) and protein content is obtained with the BCA method (Micro BCA Protein Assay Kit, ThermoFisher Scientific, Cat # 23235). The assay is described in FIG. 13.

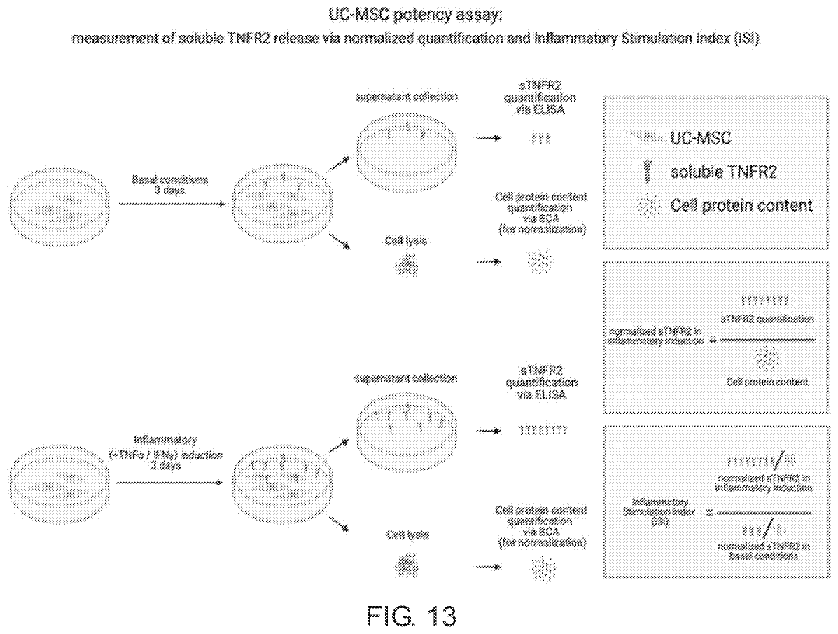

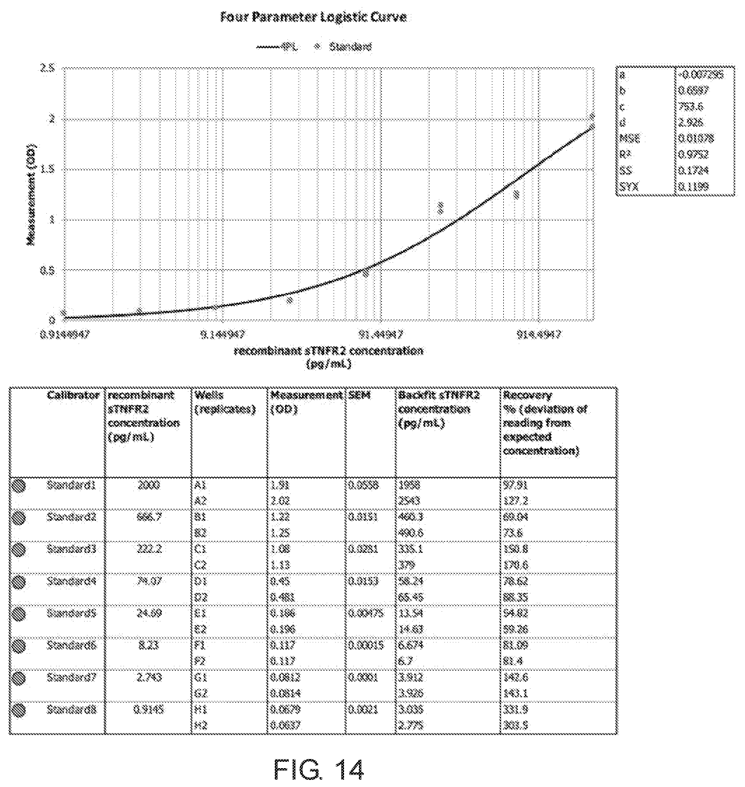

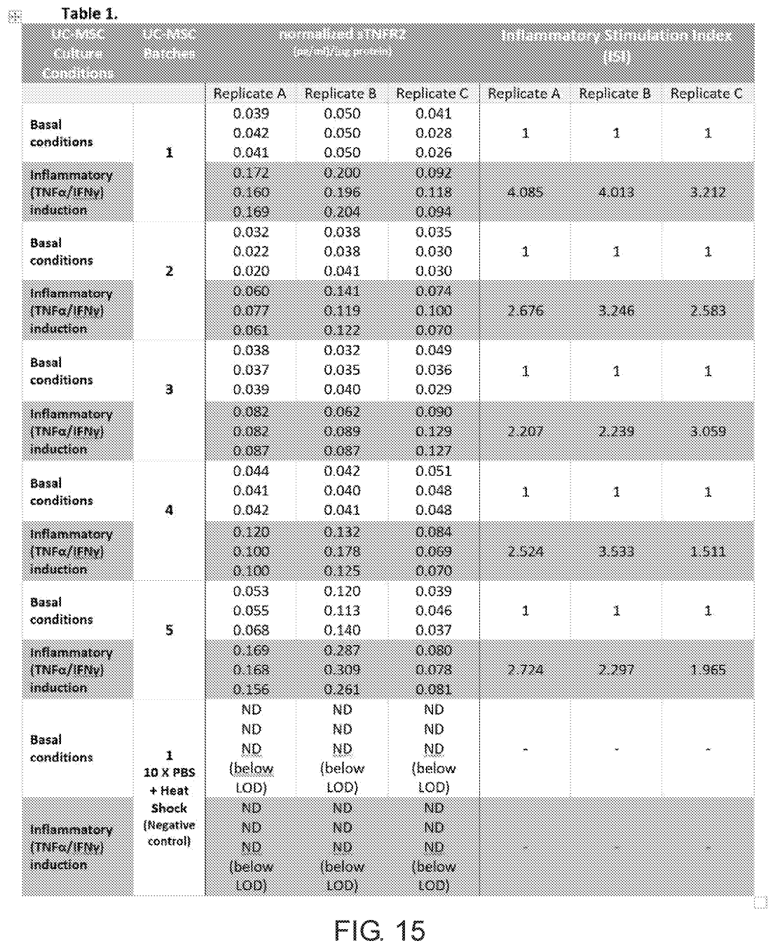

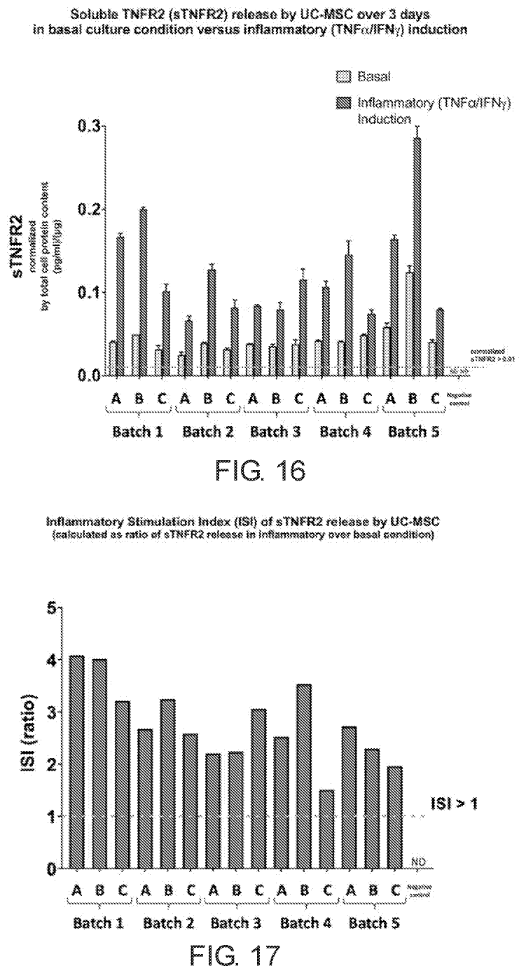

sTNFR2 release by UC-MSC over 3 days in basal culture condition versus inflammatory (TNFα/IFNγ) induction was quantified. UC-MSC used in the study were derived from 5 different batches from the same Master Cell Bank, collected and cryopreserved at different timepoints (Batch 1: 12.21.2018, Batch 2: 05.18.2020, Batch 3: 05.29.2020, Batch 4: 12.16.2020, Batch 5: 10.19.2020). From each batch, we have prepared 3 biological replicates (A, B, C) and from each replicate have obtained readouts in triplicate. For negative control, UC-MSC were treated prior to inflammatory induction with 10× PBS for 30 mins at 58° C. (Heat Shock) in order to impair their functionality and capacity to secrete sTNFR2 upon induction. The positive control for the assay is represented by the calibration curve, obtained with recombinant sTNFR2 (FIG. 14). FIG. 14 shows a calibration curve (Four Parameter Logistic Curve) based on recombinant sTNFR2. The data reported here indicates a consistent response of the UC-MSC batches by increased sTNFR2 secretion upon exposure to inflammatory mediators in vitro. This data also supports the stability and functionality of UC-MSC of different batches from the same master cell bank and cryopreserved at different timepoints over two years. This can be interpreted as the listed batches of UC-MSC maintain potency when properly cryopreserved, and that this assay indicates stability. Results are presented in Table 1 (FIG. 15), FIG. 16, and FIG. 17.

### Biological Relevance of the Assay in Relation to the Observations in Patients

The hyperinflammatory response in COVID-19 patients with Acute Distress Respiratory Syndrome (ARDS) is characterized by high serum levels of pro-inflammatory mediators, including tumor necrosis factor (TNF) α and β. These two molecules, implicated in ARDS pathophysiology, bind to TNFR2. A soluble form of TNFR2 was found to have inhibitory effect on TNF functions.

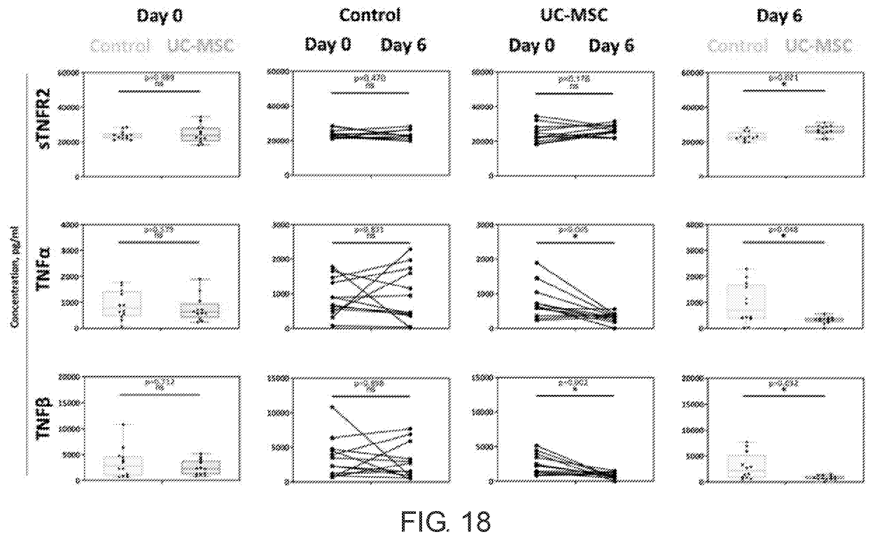

To help explain the clinical results observed with UC-MSC in the Phase 1/2a clinical trial for COVID-19 ARDS, we investigated the plasma levels of TNFα, TNFβ, and soluble TNFR2 (sTNFR2) in both UC-MSC treatment and control groups. We observed that sTNFR2 was increased in patients of the UC-MSC treatment group, compared to patients in the control group, at day 6 (see FIG. 18). Interestingly, TNFα and TNFβ were found to be decreased at day 6. The observations are presented in FIG. 18.

### METHODS (Observations in Patients)

Blood samples were obtained from clinical trial randomized subjects at day 0 (before infusion) and day 6 (3 days after second infusion). Briefly, whole blood was collected into EDTA-treated tubes, transferred on ice, and processed for plasma separation within 2 hours. Whole blood was centrifuged at 2,000 g for 15 min at 4° C., and plasma was collected and stored at —80° C. until processing. A quantitative multiplex protein array (RayBio® Q-Series, RayBiotech) was utilized to determine the TNFR2, TNFα, TNFβ plasma levels (pg/ml) in all samples at the same time, following manufacturer's instructions. The fluorescent signals were visualized via a Cy3 wavelength laser scanner and converted to concentrations using the standard curve generated per array.

Statistical analysis was performed using two sample T-tests and nonparametric Wilcoxon two-sample tests. Signed rank tests were used for paired comparisons examining changes between timepoints within group. All tests were two-sided, with statistical significance established with p<0.05. Data are presented with means and standard errors of the mean.

### RESULTS (Observations in Patients)

Patients in UC-MSC and control groups showed no significant differences in baseline protein levels. In control group, sTNFR2, TNFα and TNFβ levels were not significantly different between days 0 and 6. TNFα and TNFβ levels decreased significantly between day 0 and day 6, (p=0.005 and p=0.002, respectively). Comparisons between groups on day 6 demonstrated significantly lower levels in UC-MSC group compared to control group of TNFα (319±40 vs 950±226 pg/ml, p=0.048) and TNFβ (810±126 vs 2,944±735 pg/ml, p=0.032). sTNFR2 showed significantly higher levels in the UC-MSC group compared to control on day 6 (26,609±846 pg/ml vs 23,111±760 pg/ml, p=0.021). See FIG. 18.

FIG. 18 shows plasma concentrations of soluble tumor necrosis factor receptor 2 (sTNFR2), tumor necrosis factor alpha (TNFα), and tumor necrosis factor beta (TNFβ) in subjects with COVID-19 acute respiratory distress syndrome (ARDS) (n=24). At day 6, UC-MSC recipients had significantly elevated levels of plasma sTNFR2 and significantly decreased levels of TNFα and TNFβ compared to controls. Data are presented as box and whiskers plots indicating the median values and min to max values, and as scatter plots with lines indicating individual values.

### DISCUSSION (Observations in Patients)

In our recently completed phase 1/2a clinical trial, UC-MSC treatment was associated with accelerated clinical recovery in patients with COVID-19 ARDS. Herein, we provide molecular evidence of differences in a key underlying immune/inflammatory mediator axis that help explain those results. At day 6, UC-MSC recipients had significantly elevated levels of plasma sTNFR2 and significantly decreased levels of TNFα and TNFβ compared to controls. TNF receptor-based drugs have been tested to treat chronic inflammatory diseases, and similarly could be beneficial for the hyperinflammation attenuation in severe COVID-19 patients. TNF blockade is clinically effective as it results in rapid reduction of circulating interleukin (IL)-1 and IL-6 levels (<12 hours), and reduction in adhesion molecules and vascular endothelial growth factor (VEGF) that strongly affect leukocutes trafficking and capillary permeability in inflamed tissues (studies reviewed in). Interestingly, studies showed that upon anti-TNF therapy, TNF concentration in inflamed tissues is reduced as it passes into blood circulation bound to the anti-TNF antibodies.

Furthermore, sTNFR2 is capable of binding TNF and neutralize TNF-induced cytotoxicity and immune-reactivity, modulating inflammatory reactions. For instance, higher sTNFR2 levels lead to decreased T cell activation and gradual production of regulatory T cells (Tregs). On this basis, studies showed that expression of TNFR2 by MSC is correlated to their higher Foxp3+T reg induction capacity. Therefore, our findings could represent a key mechanism of UC-MSC effect, whereas sTNFR2 blood plasma levels may become a predictor for COVID-19 ARDS progression and clinical outcome after therapy.

### Assurance of Potency

This potency assay, measurement of soluble TNFR2 release via normalized quantification and Inflammatory Stimulation Index (ISI), is utilized for assurance of potency of the product to be used in the proposed Phase 2b/3 study. The criteria for assurance of potency of each batch of UC-MSC utilized for the Phase 2b/3 study are shown in Formula (I): soluble TNFR2 (sTNFR2) release, normalized by total cell protein content, over 3 days culture >0.01 (pg/mL)/(ug total cell protein)

Inflammatory Stimulation Index (ISI)>1

### (I)

### Justification for the Proposed Specifications

The potency assay analyzes UC-MSC's function of soluble TNFR2 (sTNFR2) release. The choice of this potency assay is supported by observations made in subjects enrolled in our Phase 1/2a clinical trial: compared to controls, subjects treated with UC-MSC presented increased levels of plasma sTNFR2 and decreased levels of TNFα, TNFβ on day 6 post infusion. The release of sTNFR2 appears to be one of the key immunomodulatory functions of UC-MSC, because sTNFR2 can modulate TNFα and β, master regulators of inflammation.

The proposed specification for sTNFR2 release is based on the observation that UC-MSC derived from the master cell bank in use release consistently more than 0.01 (pg/mL)/(ug total cell protein) over 3 days culture.

In addition, sTNFR2 was not detected in the supernatant of degraded UC-MSC, i.e. UC-MSC rendered inactive post thawing via treatment with 10× PBS and heat shock, as shown in Table 1 and FIG. 15 (negative control).

The ISI is utilized to test whether the cells can respond to an inflammatory microenvironment acquiring an anti-inflammatory phenotype, i.e. increasing their secretion of sTNFR2 compared to basal condition. Such is relevant because after infusion in recipients the cells will be exposed to an inflammatory environment. The calculation of a stimulation index to assess potency is conceptually related to that utilized for clinical lots of pancreatic islet cells (allogeneic).

The combination of normalized amount of sTNFR2 released and ISI can be therefore considered indicators of both functionality and potency of the investigational product.

The results of a double-blind RCT of UC-MSC infusion in patients hospitalized with COVID-19 and ARDS are shown above. It should be noted that, while the present disclosure describes SARS-CoV-2 infections, the compositions and therapies disclosed herein apply to any other pathogenic infection and/or any associated complications that are treatable therewith, including, without limitation, respiratory infection, ARDS conditions, complications associated with the hyper-immune, hyper-inflammatory, thrombotic responses, and the like.

### EXAMPLES

The following examples pertain to specific embodiments and point out specific features, elements, or steps that can be used or otherwise combined in achieving such embodiments.

An example is provided of a method of treating a respiratory condition in a subject, including infusing a composition comprising stem or progenitor cells to a subject having a respiratory condition, wherein the stem or progenitor cells express at least three cell markers selected from the group consisting of CD29, CD73, CD90, CD166, SSEA4, CD9, CD44, CD146, or CD105 and wherein the stem or progenitor cells do not express at least five cell markers selected from the group consisting of NANOG, CD45, CD34, CD14, CD79, CD106, CD86, CD80, CD19, CD117, Stro-1, or HLA-DR.

In another example, infusing the composition further comprises infusing the composition intravenously, intraarterially, intranasally, intraperitoneally, or a combination thereof.

In another example, infusing the composition further comprises infusing the composition intravenously, intraarterially, or a combination thereof.

In another example, infusing the composition further comprises infusing the composition intravenously.

In another example, the respiratory condition further comprises chickenpox, coronavirus infections, viral infections, non-viral infections, diphtheria, group A streptococcus, haemophilus influenzae type b, influenza, legionnaires' disease, measles, Middle East Respiratory Syndrome (MERS), mumps, pneumonia, pneumococcal meningitis, rubella, Severe Acute Respiratory Syndrome (SARS), tuberculosis, whooping cough, Acute Respiratory Distress Syndrome (ARDS), or a combination thereof.

In another example, the respiratory condition further comprises coronavirus infections, viral infections, non-viral infections, haemophilus influenzae type b, influenza, Middle East Respiratory Syndrome (MERS), pneumonia, pneumococcal meningitis, Severe Acute Respiratory Syndrome (SARS), tuberculosis, whooping cough, Acute Respiratory Distress Syndrome (ARDS), or a combination thereof.

In another example, the respiratory condition further comprises coronavirus infections, viral infections, non-viral infections, Middle East Respiratory Syndrome (MERS), pneumonia, Severe Acute Respiratory Syndrome (SARS), Acute Respiratory Distress Syndrome (ARDS), or a combination thereof.

In another example, wherein the respiratory condition further comprises Acute Respiratory Distress Syndrome (ARDS).

In another example, the stem or progenitor cells express CD29, CD73, CD90, CD166, SSEA4, CD9, CD44, CD146, and CD105.

In another example, the stem or progenitor cells do not express CD45, CD34, CD14, CD79, CD106, CD86, CD80, CD19, CD117, Stro-1, and HLA-DR.

In another example, the stem or progenitor cells are positive for SOX2.

In another example, the stem or progenitor cells are positive for OCT4.

In another example, the stem or progenitor cells are positive for SOX2 and OCT4.

In another example, the progenitor cells include a cell type selected from the group consisting of adipocytes, chondrocytes, osteocytes, cardiomyocytes, endothelial cells, mesenchymal stem cells, and myocytes.

In another example, the progenitor cells are mesenchymal stem cells.

In another example, the progenitor cells are chondrocyte cells.

In another example, the progenitor cells are osteocyte cells.

In another example, the progenitor cells are cardiomyocyte cells.

An example is provided of a kit for treating a respiratory condition in a subject, including an isolated population of stem or progenitor cells in a first container and a dilution buffer in a second container, wherein the isolated population of stem or progenitor cells express at least three cell markers selected from the group consisting of CD29, CD73, CD90, CD166, SSEA4, CD9, CD44, CD146, or CD105 and wherein the stem or progenitor cells do not express at least five cell markers selected from the group consisting of NANOG, CD45, CD34, CD14, CD79, CD106, CD86, CD80, CD19, CD117, Stro-1, or HLA-DR.

In another example, the kit is cryopreserved.

In another example, the progenitor cells include a cell type selected from the group consisting of adipocytes, chondrocytes, osteocytes, cardiomyocytes, endothelial cells, mesenchymal stem cells, and myocytes.

In another example, the progenitor cells include a cell type selected from the group consisting of adipocytes, chondrocytes, osteocytes, cardiomyocytes, endothelial cells, mesenchymal stem cells, and myocytes.

In another example, the progenitor cells are mesenchymal stem cells.

In another example, the progenitor cells are chondrocyte cells.

In another example, the progenitor cells are osteocyte cells.

In another example, the progenitor cells are cardiomyocyte cells.

What is claimed is:

1. A method of treating a respiratory condition in a subject, comprising:
infusing a composition comprising stem or progenitor cells to a subject having a respiratory condition, wherein the stem or progenitor cells express at least three cell markers selected from the group consisting of CD29, CD73, CD90, CD166, SSEA4, CD9, CD44, CD146, or CD105; and
wherein the stem or progenitor cells do not express at least five cell markers selected from the group consisting of NANOG, CD45, CD34, CD14, CD79, CD106, CD86, CD80, CD19, CD117, Stro-1, or HLA-DR.

2. The method of claim 1, wherein infusing the composition further comprises infusing the composition intravenously, intraarterially, intranasally, intraperitoneally, or a combination thereof.

3. The method of claim 1, wherein infusing the composition further comprises infusing the composition intravenously, intraarterially, or a combination thereof.

4. The method of claim 1, wherein infusing the composition further comprises infusing the composition intravenously.

5. The method of claim 1, wherein the respiratory condition further comprises chickenpox, coronavirus infections, viral infections, non-viral infections, diphtheria, group A streptococcus, haemophilus influenzae type b, influenza, legionnaires' disease, measles, Middle East Respiratory Syndrome (MERS), mumps, pneumonia, pneumococcal meningitis, rubella, Severe Acute Respiratory Syndrome (SARS), tuberculosis, whooping cough, Acute Respiratory Distress Syndrome (ARDS), or a combination thereof.

6. The method of claim 1, wherein the respiratory condition further comprises coronavirus infections, viral infections, non-viral infections, haemophilus influenzae type b, influenza, Middle East Respiratory Syndrome (MERS), pneumonia, pneumococcal meningitis, Severe Acute Respiratory Syndrome (SARS), tuberculosis, whooping cough, Acute Respiratory Distress Syndrome (ARDS), or a combination thereof

7. The method of claim 1, wherein the respiratory condition further comprises coronavirus infections, viral infections, non-viral infections, Middle East Respiratory Syndrome (MERS), pneumonia, Severe Acute Respiratory Syndrome (SARS), Acute Respiratory Distress Syndrome (ARDS), or a combination thereof.

8. The method of claim 1, wherein the respiratory condition further comprises Acute Respiratory Distress Syndrome (ARDS).

9. The method of claim 1, wherein the stem or progenitor cells express CD29, CD73, CD90, CD166, SSEA4, CD9, CD44, CD146, and CD105.

10. The method of claim 1, wherein the stem or progenitor cells do not express CD45, CD34, CD14, CD79, CD106, CD86, CD80, CD19, CD117, Stro-1, and HLA-DR.

11. The method of claim 1, wherein the stem or progenitor cells are positive for SOX2.

12. The method of claim 1, wherein the stem or progenitor cells are positive for OCT4.

13. The method of claim 1, wherein the stem or progenitor cells are positive for SOX2 and OCT4.

14. The method of claim 1, wherein the progenitor cells include a cell type selected from the group consisting of adipocytes, chondrocytes, osteocytes, cardiomyocytes, endothelial cells, mesenchymal stem cells, and myocytes.

15. The method of claim 1, wherein the progenitor cells are mesenchymal stem cells.

16. The method of claim 1, wherein the progenitor cells are chondrocyte cells.

17. The method of claim 1, wherein the progenitor cells are osteocyte cells.

18. The method of claim 1, wherein the progenitor cells are cardiomyocyte cells.

19. A kit for treating a respiratory condition in a subject, comprising:
an isolated population of stem or progenitor cells in a first container; and
a dilution buffer in a second container, wherein the isolated population of stem or progenitor cells express at least three cell markers selected from the group consisting of CD29, CD73, CD90, CD166, SSEA4, CD9, CD44, CD146, or CD105; and
wherein the stem or progenitor cells do not express at least five cell markers selected from the group consisting of NANOG, CD45, CD34, CD14, CD79, CD106, CD86, CD80, CD19, CD117, Stro-1, or HLA-DR.

20. The kit of claim 19, wherein the kit is cryopreserved.

21. The kit of claim 19, wherein the progenitor cells include a cell type selected from the group consisting of adipocytes, chondrocytes, osteocytes, cardiomyocytes, endothelial cells, mesenchymal stem cells, and myocytes.

22. The method of claim 1, wherein the progenitor cells are mesenchymal stem cells.

23. The method of claim 1, wherein the progenitor cells are chondrocyte cells.

24. The method of claim 1, wherein the progenitor cells are osteocyte cells.

25. The method of claim 1, wherein the progenitor cells are cardiomyocyte cells.
